SEQUENCE LISTING (11 sequences)

SEQ ID NO: 1 - DNA, 1701 bp, Botrytis gemydayravirus
ctatcaacaccctataattacagcgccttacaggctatcactataacaccctgaagtttcagggtctcaccgctatatttacaggctttacaggctttaaatcgctaacactaacagtgacaacaggtttcggatcagggcgcaacgtttaatgttgacctatgcccagatagatgacacgtttgacaccgaaaacttcgggccatgggtgaagaaaaagtgtggagctctgactataagagtagccctcgaggagcacaaacaaactggtggtctacacatccacgcctacgtagaagcacctacacaattcacgataaactcatccgactttctcgattactgcaaccaccatccaaacatactgccaatcagagtcacgttttacaaaacttgggattacgtaggcaaagacaacaacatcatctttgaagaagggccgccgccaccacgacctgcagaaaaatcaggatcagctgccgtgtggactgccatcgtaatgtcagccaatacaatcgacgagtttttggaggagtccttcaaagcaaggcctgggaacttgattaaaaacttcacccaattcaaggcgttcgcagagtggagatacaagccaaaggggttggaatacgtgtcaccagccattgagtgtcacatggaagattacccacaacttgagcaatgggtaatggaaaacttacgtggaggactgccagcgggtgcccgcaagcagtcattggttatgtggggtgatacaaggctgggcaaaaccctttgggctaggtcgttgggcaagcacgcatacttcccaggtatgtttatgttggacggtttcagcgaggagggctgcgagtatgccatcttcgatgacataatcgaaggattcaaaggcatcaacagttacaaggggtggtttgggtctcaacacgaggtggtcgtcaccgacaagtaccgtgggaagaggagaatcacgtggggaagaccttcggtcttcatcagcaacactgatccacgtgacgacttgcctcgagaccaagtcaagtggttagagggaaattgtgtttttgtacatattgataaagtgctatgcaggccttatgttggtgattcacctacataggcgcccaaaaaataattaattgtttccttgtcatgtgacttcgtcccattcactcacaatatttgctataaagtgtggtcatgtccaaacgcaaatgaagagtaaaattttaagacaacaagttgtcctactcttcatttgctgctgccttaacgttttttgtggaatatgcctaaacgaacctatagcgaaagggaagaaacccctggaagcatcgctggttttatcgatgatcaggcggagctttcaggctccgatgtggctgaagacccggaagacgccgatatccaagccccaaagaggcgcaaacagtgagtcactgcctatttttttaagatgtgcaacaaaaccccacagggtgtttcaggcacgtgtcaatcccgtgatacgctgaagcagcctaatcgttcaggctatgtgcgctcgatccctccctcgtgcctcggggggagcccatctcgctcaaggaccaaaactggtgaaccggaggtggtgtttattgtgaagacgcactcgctcccaggtcgctctgctcccttcccggcgggggggcccctacccctcccttacgtgctagttatagg

SEQ ID NO: 2 - protein, 321 aa, Botrytis gemydayravirus
MLTYAQIDDTFDTENFGPWVKKKCGALTIRVALEEHKQTGGLHIHAYVEAPTQFTINSSDFLDYCNHHPNILPIRVTFYKTWDYVGKDNNIIFEEGPPPPRPAEKSGSAAVWTAIVMSANTIDEFLEESFKARPGNLIKNFTQFKAFAEWRYKPKGLEYVSPAIECHMEDYPQLEQWVMENLRGGLPAGARKQSLVMWGDTRLGKTLWARSLGKHAYFPGMFMLDGFSEEGCEYAIFDDIIEGFKGINSYKGWFGSQHEVVVTDKYRGKRRITWGRPSVFISNTDPRDDLPRDQVKWLEGNCVFVHIDKVLCRPYVGDSPT

SEQ ID NO: 3 - DNA, 966 bp, Botrytis gemydayravirus
atgttgacctatgcccagatagatgacacgtttgacaccgaaaacttcgggccatgggtgaagaaaaagtgtggagctctgactataagagtagccctcgaggagcacaaacaaactggtggtctacacatccacgcctacgtagaagcacctacacaattcacgataaactcatccgactttctcgattactgcaaccaccatccaaacatactgccaatcagagtcacgttttacaaaacttgggattacgtaggcaaagacaacaacatcatctttgaagaagggccgccgccaccacgacctgcagaaaaatcaggatcagctgccgtgtggactgccatcgtaatgtcagccaatacaatcgacgagtttttggaggagtccttcaaagcaaggcctgggaacttgattaaaaacttcacccaattcaaggcgttcgcagagtggagatacaagccaaaggggttggaatacgtgtcaccagccattgagtgtcacatggaagattacccacaacttgagcaatgggtaatggaaaacttacgtggaggactgccagcgggtgcccgcaagcagtcattggttatgtggggtgatacaaggctgggcaaaaccctttgggctaggtcgttgggcaagcacgcatacttcccaggtatgtttatgttggacggtttcagcgaggagggctgcgagtatgccatcttcgatgacataatcgaaggattcaaaggcatcaacagttacaaggggtggtttgggtctcaacacgaggtggtcgtcaccgacaagtaccgtgggaagaggagaatcacgtggggaagaccttcggtcttcatcagcaacactgatccacgtgacgacttgcctcgagaccaagtcaagtggttagagggaaattgtgtttttgtacatattgataaagtgctatgcaggccttatgttggtgattcacctacatag

SEQ ID NO: 4 - protein, 124 aa, Botrytis gemydayravirus
MFPCHVTSSHSLTIFAIKCGHVQTQMKSKILRQQVVLLFICCCLNVFCGICLNEPIAKGKKPLEASLVLSMIRRSFQAPMWLKTRKTPISKPQRGANSESLPIFLRCATKPHRVFQARVNPVIR

SEQ ID NO: 5 - DNA, 375 bp, Botrytis gemydayravirus
ttgtttccttgtcatgtgacttcgtcccattcactcacaatatttgctataaagtgtggtcatgtccaaacgcaaatgaagagtaaaattttaagacaacaagttgtcctactcttcatttgctgctgccttaacgttttttgtggaatatgcctaaacgaacctatagcgaaagggaagaaacccctggaagcatcgctggttttatcgatgatcaggcggagctttcaggctccgatgtggctgaagacccggaagacgccgatatccaagccccaaagaggcgcaaacagtgagtcactgcctatttttttaagatgtgcaacaaaaccccacagggtgtttcaggcacgtgtcaatcccgtgatacgctga

SEQ ID NO: 6 - protein, 97 aa, Botrytis gemydayravirus
MCNKTPQGVSGTCQSRDTLKQPNRSGYVRSIPPSCLGGSPSRSRTKTGEPEVVFIVKTHSLPGRSAPFPAGGPLPLPYVLVIGYQHPIITAPYRLSL

SEQ ID NO: 7 - DNA, 294 bp, Botrytis gemydayravirus
atgtgcaacaaaaccccacagggtgtttcaggcacgtgtcaatcccgtgatacgctgaagcagcctaatcgttcaggctatgtgcgctcgatccctccctcgtgcctcggggggagcccatctcgctcaaggaccaaaactggtgaaccggaggtggtgtttattgtgaagacgcactcgctcccaggtcgctctgctcccttcccggcgggggggcccctacccctcccttacgtgctagttataggctatcaacaccctataattacagcgccttacaggctatcactataa

SEQ ID NO: 8 - DNA, 20 bp, Artificial Sequence
  Primer
ggagatacaagccaaagggg

SEQ ID NO: 9 - DNA, 20 bp, Artificial Sequence
  Primer
ctgtttgcgcctctttgggg

SEQ ID NO: 10 - DNA, 22 bp, Artificial Sequence
  Primer
ctactcttcatttgctgctgcc

SEQ ID NO: 11 - DNA, 20 bp, Artificial Sequence
  Primer
cttgcccaacgacctagccc

(19) United States
(12) Patent Application Publication
(10) Pub. No.: US 2020/0323218 A1
(43) Pub. Date: Oct. 15, 2020

(54) Viral Mediated Biological Control of Plant Pathogenic Microorganisms

(71) Applicant: The New Zealand Institute for Plant and Food Research Limited, Auckland (NZ)

(72) Inventors: Mahmoud Elhoseny Elhoseny Elsayed KHALIFA, Damietta (EG); Robin Marion MACDIARMID, Kerikeri (NZ)

(73) Assignee: The New Zealand Institute for Plant and Food Research Limited, Auckland (NZ)

(21) Appl. No.: 16/956,296

(22) PCT Filed: Dec. 20, 2018
(86) PCT No.: PCT/IB2018/060395

§ 371 Date: Jun. 19, 2020

(30) Foreign Application Priority Data
Dec. 21, 2017 (AU) 2017905109

(51) Int. Cl. A01N 63/40; C12R 1/91; C07K 14/005; C12N 7/00

(52) U.S. Cl. CPC A01N 63/40; C12R 1/91; C07K 14/005; C12N 2795/10143; C12N 2795/18021; C12N 2795/18022; C12N 2795/18031; C12N 7/00

(57) ABSTRACT

The present invention relates to an isolated strain of DNA mycovirus or a degenerate strain thereof, an isolated hypovirulent fungal strain or part thereof, and the use of the isolated strain of DNA mycovirus or hypovirulent fungal strain or part thereof as a biological control agent. Processes and compositions for the biological control of phytopathogenic microorganisms, particularly fungi, using the DNA mycovirus and hypovirulent fungal strain are also provided.

### FIELD OF THE INVENTION

This invention relates generally to methods of using viruses, particularly mycoviruses, for the biological control of plant pathogenic microorganisms, particularly fungi. In particular, the invention relates to a novel mycovirus strain having biological control activity, and to methods of using such to inhibit the survival, growth and/or proliferation of plant pathogenic microorganisms, particularly fungi, on plants or parts thereof.

### BACKGROUND OF THE INVENTION

Botrytis cinerea, unlike the majority of other Botrytis species that are restricted to certain hosts, is a ubiquitous ascomycetious phytopathogen (Elad et al. 1996) capable of infecting a wide range of host species in New Zealand and worldwide. The fungus was reported to have over 100 hosts in New Zealand (Pennycook 1989) and over 230 hosts worldwide (Jarvis 1977). It causes several pre- and post-harvest diseases including grey mould, leaf blight, blossom blight, bunch rot disease, and post-harvest fruit rots (Jarvis 1977; Elad et al. 2004), with the grey mould being the most common.

Worldwide, it is estimated that the grape crop losses from B. cinerea, the causal agent of Botrytis bunch rot which reduces the quality and quantity of the yield (Bulit & Dubos 1988), is up to 2 billion USD annually (Elmer & Michailides 2007). In 2002, $NZ9.9 million was the estimated loss to the grape crop with potential loss to the wine industry valued at $NZ49 million (Beresford 2005). B. cinerea diseases are most commonly controlled chemically through the application of fungicides. However, this practice of fungicide application is of increasing concern due to their high cost, their hazardous impact on the environment (Rocha & Oliveira 1998) and the ability of host fungi to develop resistance to fungicides (Williamson et al. 2007). Accordingly there is a need for other means of fungal disease control.

It is an object of the invention to provide at least one viral biological control agent and/or a composition comprising at least one viral biological control agent and/or methods of using such an agent and/or such a composition for controlling at least one plant pathogenic fungus on at least one plant or part thereof, preferably wherein the fungus is Botrytis spp.; and/or to at least to provide the public with a useful choice.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

### SUMMARY OF THE INVENTION

In one aspect the invention relates to an isolated DNA mycovirus or degenerate strain thereof encoding at least one polypeptide comprising at least 70% amino acid sequence identity to a polypeptide selected from the group consisting of SEQ ID NO: 2, SEQ ID NO: 4 and SEQ ID NO: 6.

In another aspect the invention relates to an isolated polypeptide comprising at least 70% amino acid sequence identity to an amino acid sequence selected from the group consisting of SEQ ID NO: 2, SEQ ID NO: 4 and SEQ ID NO: 6.

In another aspect the invention relates to an isolated nucleic acid sequence encoding an isolated polypeptide of the invention.

In another aspect the invention relates to an isolated nucleic acid sequence comprising at least 70% nucleotide sequence identity to SEQ ID NO: 1.

In another aspect the invention relates to an isolated DNA mycovirus comprising a nucleic acid sequence comprising at least 70% nucleotide sequence identity to SEQ ID NO: 1 or a degenerate strain thereof.

In another aspect the invention relates to an isolated DNA mycovirus comprising SEQ ID NO: 1, or a degenerate strain thereof.

In another aspect the invention relates to a vector comprising a nucleic acid sequence according to the invention.

In another aspect the invention relates to an isolated host cell comprising an isolated nucleic acid sequence, vector, polypeptide or DNA mycovirus or degenerate strain thereof of the invention.

In another aspect the invention relates to a hypovirulent fungal strain or part thereof comprising an isolated nucleic acid sequence, vector, polypeptide or DNA mycovirus or degenerate strain thereof of the invention.

In another aspect the invention relates to a composition comprising an isolated nucleic acid sequence, vector, polypeptide, DNA mycovirus or degenerate strain thereof, isolated host cell, hypovirulent fungal strain or part thereof, or a combination thereof, of the invention, and a carrier, diluent or excipient.

In another aspect the invention relates to a method of reducing the virulence of at least one phytopathogenic fungus comprising contacting the fungus with an isolated DNA mycovirus of the invention, or a degenerate strain thereof.

In another aspect the invention relates to a method of Botrytis spp. biocontrol comprising contacting at least one Botrytis spp. with an isolated DNA mycovirus, or degenerate strain thereof.

In another aspect the invention relates to a method of treating at least one plant disease caused by a phytopathogenic fungus comprising contacting the plant with an isolated DNA mycovirus or degenerate strain thereof of the invention or a hypovirulent fungal strain or part thereof of the invention, or both.

In another aspect the invention relates to a method of controlling at least one phytopathogenic fungus comprising contacting the fungus with an isolated DNA mycovirus or degenerate strain thereof of the invention or a hypovirulent fungal strain or part thereof of the invention, or both.

In another aspect the invention relates to an isolated DNA mycovirus or degenerate strain thereof of the invention for use in controlling at least one phytopathogenic fungal strain.

In another aspect the invention relates to an isolated hypovirulent fungal strain or part thereof of the invention for use in controlling at least one phytopathogenic fungal strain.

In another aspect the invention relates to an isolated DNA mycovirus, or a degenerate strain thereof, for use in controlling Botrytis spp. fungi.

While various embodiments of certain aspects of the invention may be set out above, the invention is not limited thereto. Additional embodiments of the aspects of the invention set out above are further described in the Detailed Description and set out in the claims of the application.

Other aspects and embodiments of the invention may become apparent from the following description which is given by way of example only and with reference to the accompanying drawings.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

### BRIEF DESCRIPTION OF THE DRAWINGS

The invention will now be described by way of example only and with reference to the drawings in which:

FIG. 1. PCR detection of Botrytis gemydayravirus 1 (BGDaV1), suggested name according to the nomenclature for the family Genomoviridae established in Varsani and Krupovic, 2017, Virus Evolution. 3(1):vew037) in different DNA pools. M: 1 kb⁺ DNA molecular weight marker (Invitrogen); W: water negative control.

FIG. 2 (A) Virus particles observed under transmission electron microscopy from isolate 339-13. Bar=200 nm. (B) EcoRV-digested RCA products of circular DNA associated with isolates 339-13 and 339-42. (C) Viral nucleic acid (DNA) isolated from purified virus like particles (VLPs) of isolate 339-13. (D) Schematic illustration of the genome organisation of BGDaV1. (E) Rep AA sequence alignment of BGDaV1 and other circular ssDNA sequences for the identification of BGDaV1 Rep conserved motifs.

FIG. 3 Phylogenetic relationship between BGDaV1 and other selected circular ssDNA viruses. Multiple sequence alignment of the deduced AA sequences of the Rep was conducted using MUSCLE. The maximum likelihood tree was displayed using MEGA 7 software using LG model combined with gamma-distributed rates across sites. The results of bootstrapping analysis of 100 replicates are indicated by numbers on the branches.

FIG. 4 DsRNA profile of BGDaV1-containg isolates. M: 1 kb⁺ DNA molecular weight marker (Invitrogen).

FIG. 5 Lesion diameter comparisons between differently treated Botrytis cinerea isolates developed on detached leaves of canola. In treatment 702, mycelial plugs of the virus-free isolate 702 were used to inoculate canola detached leaves. In treatments 702-V101 and 702-V49, the virus free isolate 702 was mechanically inoculated with VLPs purified from isolates 339-101 and 339-49, respectively, and the newly-infected progeny were used to inoculate canola detached leaves. In treatment 702-Vmix, a drop of VLPs mixture purified from fungal isolates 339-13, 339-49 and 339-101 was applied on canola detached leaves before they were inoculated with mycelial plugs of virus-free isolate 702. Lesion diameter measurements were taken after a 4- to 5-day incubation period of three replicates in each treatment. Different letters indicate significantly different (P<0.050) treatments.

FIG. 6 Examples of growth of Botrytis cinerea infected or not with BGDaV1 after 4 days inoculation (Assay 1) or 5 days inoculation (Assay 2) on cyclamen leaves with a plug of either potato dextrose agar (PDA) or inoculum grown on PDA. PDA inoculations result in no disease. Botrytis, virus-free (B. cinerea only) result in disease symptoms including brown discoloration of leaf tissue beyond the margin of the inoculation plug (Assay 1) or within the inoculation plug (Assay 2). Botrytis, 21918, Botrytis 21919, Botrytis 21220 and Botrytis 21921 result in reduced disease expression, especially in Assay 1, strains Botrytis 21918 and Botrytis 21919.

FIG. 7 Examples of growth of Botrytis cinerea infected or not with BGDaV1 after 6 days inoculation (Assay 1) on strawberry leaves (two cultivars) with a plug of either potato dextrose agar (PDA) or inoculum grown on PDA. Only one leaf is shown for each cultivar. PDA inoculations result in no botrytis growth. Botrytis, virus-free (B. cinerea only) result in prolific white mycelium growth beyond the margin of the inoculation plug (more than 1 cm) that at times reached the edge leaf. Botrytis, 21918, Botrytis 21919, Botrytis 21220 and Botrytis 21921 result in reduced botrytis growth; either no botrytis mycelium growth was observed beyond the inoculation plug or botrytis mycelium growth reached less than a maximum of 0.5 cm from the inoculation plug.

FIG. 8 Examples of growth of Botrytis cinerea infected or not with BGDaV1 after 5 days inoculation (Assay 2) on kiwifruit leaves with a plug of inoculum grown on PDA either virus-free or infected with BGDaV1. PDA inoculations result in no botrytis growth. Botrytis, virus-free (B. cinerea only) result in some botrytis mycelium growth and brown discoloured lesions around the inoculation plug. Botrytis, 21918, Botrytis 21919, Botrytis 21220 and Botrytis 21921 result in little or no visible lesion around the inoculation plug.

FIG. 9 Examples of growth of Botrytis cinerea infected or not with BGDaV1 after 4 days inoculation (Assay 1) on A) grape berries (either cut or not cut) with a plug of either potato dextrose agar (PDA) or inoculum grown on PDA. Three un-inoculated grapes were added to the cut grape assay to identify any contamination (grey boxes). B), To assess penetration of B. cinerea into berries they were cut in half 7 days post inoculation (dpi). Arrow indicates positon of integrity loss within the grape berry. In Assay 1, BGDaV1-infected B. cinerea resulted in slower growth than virus-free B. cinerea, particularly when the table grapes were not pre-cut and isolates were infected with BGDaV1 21918, 4 dpi (FIG. 3A). Furthermore, when the grapes were cut in half at 7 dpi, the grapes inoculated with the virus-free B. cinerea isolate generally had loose grape integrity (a similar phenotype to botrytis bunch rot), grapes were softer, and considerably misshaped compared with grapes inoculated with BGDaV1-infected B. cinerea, which were harder and retained their shape.

FIG. 10 Examples of growth of Botrytis cinerea infected or not with BGDaV1 after 4 days inoculation (Assay 2) on A), grape berries (either cut or not cut) with a plug of either potato dextrose agar (PDA) or inoculum grown on PDA. Un-inoculated grapes were placed between treated grapes (grey boxes). B), To assess penetration of B. cinerea into berries they were cut in half at 7 dpi. Arrow indicates positon of integrity loss within the grape berry. Virus-free B. cinerea-inoculated grapes lost shape when they were cut in half. By comparison, grapes inoculated with virus-infected B. cinerea (Botrytis 21918, 21919, and 21920) retained their shape and firm texture.

### DETAILED DESCRIPTION OF THE INVENTION

### Definitions

The following definitions are presented to better define the present invention and as a guide for those of ordinary skill in the art in the practice of the present invention.

Unless otherwise specified, all technical and scientific terms used herein are to be understood as having the same meanings as is understood by one of ordinary skill in the relevant art to which this disclosure pertains.

Examples of definitions of common terms in botany, microbiology, molecular biology and biochemistry can be found in Biology of Plants, Raven et al. (eds.), W.H. Freeman and Company, (2005); Plant Physiology, Taiz et al. (eds.), Sinauer Associates, Incorporated, (2010); Botany: An Introduction to Plant Biology, J. D. Mauseth, Jones & Bartlett Learning, (2003); Methods for General and Molecular Microbiology, 3rd Edition, C. A. Reddy, et al. (eds.), ASM Press, (2008); Encyclopedia of Microbiology, 2nd ed., Joshua Lederburg, (ed.), Academic Press, (2000); Microbiology By Cliffs Notes, I. Edward Alcamo, Wiley, (1996); Dictionary of Microbiology and Molecular Biology, Singleton et al. (2d ed.) (1994); Biology of Microorganisms 11th ed., Brock et al., Pearson Prentice Hall, (2006); Biodiversity of Fungi: Inventory and Monitoring Methods, Mueller et al., Academic Press, (2004); Genes IX, Benjamin Lewin, Jones & Bartlett Publishing, (2007); The Encyclopedia of Molecular Biology, Kendrew et al. (eds.), Blackwell Science Ltd., (1994); and Molecular Biology and Biotechnology: a Comprehensive Desk Reference, Robert A. Meyers (ed.), VCH Publishers, Inc., (1995).

It is also believed that practice of the present invention can be performed using standard botanical, microbiological, molecular biology and biochemistry protocols and procedures as known in the art, and as described, for example in Environmental Microbiology: Methods and Protocols, J. F. T. Spencer et al., Humana Press, (2004); Environmental Microbiology, P. D. Sharma, Alpha Science International, (2005); Environmental Microbiology, J. R. Leadbetter, Gulf Professional Publishing, (2005) and other commonly available reference materials relevant in the art to which this disclosure pertains, and which are all incorporated by reference herein in their entireties.

The term “comprising” as used in this specification means “consisting at least in part of”. When interpreting each statement in this specification that includes the term “comprising”, features other than that or those prefaced by the term may also be present. Related terms such as “comprise” and “comprises” are to be interpreted in the same manner.

The term “consisting essentially of” as used herein means the specified materials or steps and those that do not materially affect the basic and novel characteristic(s) of the claimed invention.

The term “consisting of” as used herein means the specified materials or steps of the claimed invention, excluding any element, step, or ingredient not specified in the claim.

The term “plant” as used herein encompasses whole plants and all parts of a plant from all stages of a plant lifecycle including but not limited to vegetative and reproductive cells and tissues, propagules, seeds, embryos, fruits, shoots, stems, leaves, leaf sheaths and blades, inflorescences, roots, anthers, ligules, palisade, mesophyll, epidermis, auricles, palea, lemma and tillers.

The term “biological control agent” as used herein refers to agents which act as an antagonist of one or more plant pathogens. Antagonists may take a number of forms. In one form, the biological control agent may out-compete the pathogen for available nutrients and/or space of the host plant. In another form the biological control agent may render the environment unfavourable for the pathogen. Accordingly, the antagonist mechanisms include but are not limited to hypovirulence, antibiosis, mycoparasitism, nutrient competition and physical displacement.

The terms “control”, “controlling”, “biocontrol” or “biological control” are used interchangeably herein to refer to the reduction of the amount of inoculum or disease-producing activity of a pathogen accomplished by or through one or more microorganisms. Generally comprehended is the prevention or reduction of infection by plant pathogenic bacteria or fungi, particularly plant pathogenic fungi including Botrytis spp., particularly or inhibition of the rate or extent of such infection, including any reduction in the survival, growth and/or proliferation of the bacteria or fungi. Curative treatment is also contemplated.

The term “statistically significant” as used herein refers to the likelihood that a result or relationship is caused by something other than random chance. A result may be found to be statistically significant using statistical hypothesis testing as known and used in the art. Statistical hypothesis testing provides a “P-value” as known in the art, which represents the probability that the measured result is due to random chance alone. It is believed to be generally accepted in the art that levels of significance of 5% (0.05) or lower are considered to be statistically significant.

The term “effective amount” as used herein means an amount effective to protect against, delay, reduce, stabilise, improve or treat plant pathogenic bacterial or fungal infection in and/or on a plant.

The phrase “reducing the virulence” as used herein means that the presence of the virus results in less or slower growth of the host fungus or less or slower onset of disease of the host plant, or part thereof, of the fungus, than in the absence of the virus.

The phrase “hypovirulent fungal strain or part thereof” as used herein encompasses the cells, hyphae, mycelia, conidia, sclerotia, asci and spores of the fungal strain as well as any and all parts of the cells, hyphae, mycelia, conidia, sclerotia, asci and spores of the fungal strain.

An “agriculturally acceptable adjuvant” as used herein refers to a compound or material that is generally comprehended in the art of agriculture as a useful additive in agricultural formulations or carried out with agricultural treatments.

An “additional active agent” as used herein means any compound or material that is capable of contributing to the control (as defined herein) of phytopathogenic fungi, particularly Botrytis spp. by a DNA mycovirus useful in the present invention, or that is capable of potentiating the effects of the DNA mycovirus useful in this invention in controlling plant disease caused by phytopathogenic fungi, particularly Botrytis spp., but not limited thereto.

A “formulation agent” as used herein refers to any compound or material that facilitates or optimizes the production, handling, storage, transport, application and/or persistence of the composition of, or for use in the invention on plants (as defined herein), but not limited thereto.

An “agriculturally acceptable carrier” is used herein as is generally comprehended in the art. A preferred agriculturally acceptable carrier is water, but not limited thereto.

The term “polynucleotide(s),” as used herein, means a single or double-stranded deoxyribonucleotide or ribonucleotide polymer of any length, and include as non-limiting examples, coding and non-coding sequences of a gene, sense and antisense sequences, exons, introns, genomic DNA, cDNA, pre-mRNA, mRNA, rRNA, siRNA, miRNA, tRNA, ribozymes, recombinant polynucleotides, isolated and purified naturally occurring DNA or RNA sequences, synthetic RNA and DNA sequences, nucleic acid probes, primers, fragments, genetic constructs, vectors and modified polynucleotides. Reference to nucleic acids, nucleic acid molecules, nucleotide sequences and polynucleotide sequences is to be similarly understood.

The term “vector” as used herein refers to a polynucleotide molecule, usually double stranded DNA, which is used to replicate or express a genetic construct. The vector may be used to transport a genetic construct into a given host cell.

The term “coding region” or “open reading frame” (ORF) refers to the sense strand of a genomic DNA sequence or a cDNA sequence that is capable of producing a transcription product and/or a polypeptide under the control of appropriate regulatory sequences.

The coding sequence is identified by the presence of a 5′ translation start codon and a 3′ translation stop codon. When inserted into a genetic construct or an expression cassette, a “coding sequence” is capable of being expressed when it is operably linked to promoter and terminator sequences and/or other regulatory elements.

A “functional fragment” of a polypeptide is a subsequence of the polypeptide that performs a function that is required for the biological activity or binding of that polypeptide and/or provides the three dimensional structure of the polypeptide. The term may refer to a polypeptide, an aggregate of a polypeptide such as a dimer or other multimer, a fusion polypeptide, a polypeptide fragment, a polypeptide variant, or functional polypeptide derivative thereof that is capable of performing the polypeptide activity.

“Isolated” as used herein with reference to polynucleotide or polypeptide sequences describes a sequence that has been removed from its natural cellular environment. An isolated molecule may be obtained by any method or combination of methods as known and used in the art, including biochemical, recombinant, and synthetic techniques. The polynucleotide or polypeptide sequences may be prepared by at least one purification step.

“Isolated” when used herein in reference to a cell or host cell describes to a cell or host cell that has been obtained or removed from an organism or from its natural environment and is subsequently maintained in a laboratory environment as known in the art. The term encompasses single cells, per se, as well as cells or host cells comprised in a cell culture and can include a single cell or single host cell.

The term “recombinant” refers to a polynucleotide sequence that is removed from sequences that surround it in its natural context and/or is recombined with sequences that are not present in its natural context. A “recombinant” polypeptide sequence is produced by translation from a “recombinant” polynucleotide sequence.

As used herein, the term “variant” refers to polynucleotide or polypeptide sequences different from the specifically identified sequences, wherein one or more nucleotides or amino acid residues is deleted, substituted, or added. Variants may be naturally occurring allelic variants, or non-naturally occurring variants. Variants may be from the same or from other species and may encompass homologues, paralogues and orthologues. In certain embodiments, variants of the polypeptides useful in the invention have biological activities that are the same or similar to those of a corresponding wild type molecule; i.e., the parent polypeptides or polynucleotides.

In certain embodiments, variants of the polypeptides described herein have biological activities that are similar, or that are substantially similar to their corresponding wild type molecules. In certain embodiments the similarities are similar activity and/or binding specificity.

In certain embodiments, variants of polypeptides described herein have biological activities that differ from their corresponding wild type molecules. In certain embodiments the differences are altered activity and/or binding specificity.

The term “variant” with reference to polynucleotides and polypeptides encompasses all forms of polynucleotides and polypeptides as defined herein.

Variant polynucleotide sequences preferably exhibit at least 50%, at least 60%, preferably at least 70%, preferably at least 71%, preferably at least 72%, preferably at least 73%, preferably at least 74%, preferably at least 75%, preferably at least 76%, preferably at least 77%, preferably at least 78%, preferably at least 79%, preferably at least 80%, preferably at least 81%, preferably at least 82%, preferably at least 83%, preferably at least 84%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, and preferably at least 99% identity to a sequence of the present invention. Identity is found over a comparison window of at least 8 nucleotide positions, preferably at least 10 nucleotide positions, preferably at least 15 nucleotide positions, preferably at least 20 nucleotide positions, preferably at least 27 nucleotide positions, preferably at least 40 nucleotide positions, preferably at least 50 nucleotide positions, preferably at least 60 nucleotide positions, preferably at least 70 nucleotide positions, preferably at least 80 nucleotide positions, preferably over the entire length of a polynucleotide used in or identified according to a method of the invention.

Polynucleotide variants also encompass those which exhibit a similarity to one or more of the specifically identified sequences that is likely to preserve the functional equivalence of those sequences and which could not reasonably be expected to have occurred by random chance.

Polynucleotide sequence identity and similarity can be determined readily by those of skill in the art.

Variant polynucleotides also encompasses polynucleotides that differ from the polynucleotide sequences described herein but that, as a consequence of the degeneracy of the genetic code, encode a polypeptide having similar activity to a polypeptide encoded by a polynucleotide of the present invention. A sequence alteration that does not change the amino acid sequence of the polypeptide is a “silent variation”. Except for ATG (methionine) and TGG (tryptophan), other codons for the same amino acid may be changed by art recognized techniques, e.g., to optimize codon expression in a particular host organism.

The term “degenerate sequence thereof” with reference to a nucleic acid sequence means a nucleic acid sequence variant of an initial sequence that differs from the initial sequence due only to degeneracy in the nucleic acid code.

The term “degenerate strain thereof” means an isolated DNA mycovirus strain as described herein that is a nucleic acid sequence variant of an initial DNA mycovirus strain and differs from the initial strain due 1) to degeneracy in the nucleic acid code, or 2) to nucleic acid substitutions, additions and/or deletions in non-coding regions that do not change or alter the biological functions of the virus, or 3) to nucleic acid sequence variations that encode at least one variant mycovirus polypeptide wherein the amino acid sequence of the at least one variant polypeptide in the degenerate strain differs from the amino acid sequence of the equivalent polypeptide produced by the initial mycovirus strain due to amino acid changes, particularly conservative amino acid changes, that do not change or alter the biological function(s) of the polypeptide.

Polynucleotide sequence alterations resulting in conservative substitutions of one or several amino acids in the encoded polypeptide sequence without significantly altering its biological activity are also included in the invention. A skilled artisan will be aware of methods for making phenotypically silent amino acid substitutions (see, e.g., Bowie et al., 1990, Science 247, 1306).

The term “variant” with reference to polypeptides also encompasses naturally occurring, recombinantly and synthetically produced polypeptides. Variant polypeptide sequences preferably exhibit at least 50%, preferably at least 60%, preferably at least 70%, preferably at least 71%, preferably at least 72%, preferably at least 73%, preferably at least 74%, preferably at least 75%, preferably at least 76%, preferably at least 77%, preferably at least 78%, preferably at least 79%, preferably at least 80%, preferably at least 81%, preferably at least 82%, preferably at least 83%, preferably at least 84%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, and preferably at least 99% identity to a sequence of the present invention. Identity is found over a comparison window of at least 2 amino acid positions, preferably at least 3 amino acid positions, preferably at least 4 amino acid positions, preferably at least 5 amino acid positions, preferably at least 7 amino acid positions, preferably at least 10 amino acid positions, preferably at least 15 amino acid positions, preferably at least 20 amino acid positions, preferably over the entire length of a polypeptide used in or identified according to a method of the invention.

Polypeptide variants also encompass those which exhibit a similarity to one or more of the specifically identified sequences that is likely to preserve the functional equivalence of those sequences and which could not reasonably be expected to have occurred by random chance.

Polypeptide sequence identity and similarity can be determined readily by those of skill in the art.

A variant polypeptide includes a polypeptide wherein the amino acid sequence differs from a polypeptide herein by one or more conservative amino acid substitutions, deletions, additions or insertions which do not affect the biological activity of the peptide. Conservative substitutions typically include the substitution of one amino acid for another with similar characteristics, e.g., substitutions within the following groups: valine, glycine; glycine, alanine; valine, isoleucine, leucine; aspartic acid, glutamic acid; asparagine, glutamine; serine, threonine; lysine, arginine; and phenylalanine, tyrosine.

Non-conservative substitutions will entail exchanging a member of one of these classes for a member of another class.

Analysis of evolved biological sequences has shown that not all sequence changes are equally likely, reflecting at least in part the differences in conservative versus non-conservative substitutions at a biological level. For example, certain amino acid substitutions may occur frequently, whereas others are very rare. Evolutionary changes or substitutions in amino acid residues can be modelled by a scoring matrix also referred to as a substitution matrix. Such matrices are used in bioinformatics analysis to identify relationships between sequences and are known to the skilled worker.

Other variants include peptides with modifications which influence peptide stability. Such analogs may contain, for example, one or more non-peptide bonds (which replace the peptide bonds) in the peptide sequence. Also included are analogs that include residues other than naturally occurring L-amino acids, e.g. D-amino acids or non-naturally occurring synthetic amino acids, e.g. beta or gamma amino acids and cyclic analogs.

### DETAILED DESCRIPTION OF THE INVENTION

The present invention relates generally to a novel circular single stranded (ss) DNA mycovirus isolated from Botrytis cinerea which is tentatively named as Botrytis gemydayravirus 1 (BGDaV1). BGDaV1 and compositions comprising BGDaV1 as described herein are useful for the biocontrol of plant disease caused by plant pathogenic fungi, particularly Botrytis spp. fungi. The invention also relates generally to methods of controlling phytopathogenic fungi, particularly Botrytis spp. of fungi on a plant or part thereof by contacting the plant or part thereof with BGDaV1 or a degenerate strain thereof or with a hypovirulent fungal strain, particularly a hypovirulent Botrytis spp., or part thereof

The applicants are the first to provide a DNA mycovirus that confers hypovirulence on Botrytis spp. fungi and that can be used as a biocontrol agent, and compositions comprising a DNA mycovirus and an agriculturally acceptable carrier that are effective at controlling Botrytis spp. fungi on plants. In some embodiments the DNA mycovirus is BGDaV1. In some embodiments, the DNA mycovirus is comprised in a hypovirulent fungal strain, particularly a hypovirulent Botrytis spp. strain, or part thereof. In some embodiments, the DNA mycovirus or a degenerate strain thereof or the hypovirulent fungal strain or part thereof, or both, are comprised in a composition wherein the composition is formulated with an agriculturally acceptable adjuvant.

The applicants are also the first to provide methods of using a DNA mycovirus, or a hypovirulent strain of Botrytis spp. containing a DNA mycovirus, for biological control of Botrytis spp. In particular, the applicants are the first to show that a strain of DNA mycovirus, BGDaV1, or a composition comprising BGDaV1, is effective at inhibiting the survival, growth and/or proliferation of Botrytis spp. on plants.

Without wishing to be bound by theory the applicants believe that the efficacy of the DNA mycovirus of the invention relates to the ability of the virus to confer hypovirulence to phytopathogenic fungi, particularly Botrytis spp. In some embodiments, transmission to the phytopathogenic fungus, particularly Botrytis spp. is extracellular, particularly by mechanical transmission. As used herein, mechanical transmission means that the virus is able to infect a new fungal cell through the fungal cell wall.

Irrespective of the particular mode of action, the inventors have surprisingly found that BGDaV1, hypovirulent strains of fungi containing BGDaV1, and compositions comprising BGDaV1, are efficacious for treating Botrytis spp. infection on plants and/or plant parts thereof.

BGDaV1 is a particularly effective biological control agent against Botrytis spp. fungi. BGDaV1 demonstrates the ability to survive formulation and application protocols, rapidly colonise treated plants, and suppress growth of Botrytis spp. fungi on treated plants. BGDaV1 has been found to be particularly effective at controlling Botrytis cinerea.

### DNA Mycoviruses and Compositions

Accordingly, in one aspect the invention relates to an isolated DNA mycovirus or a degenerate strain thereof encoding at least one polypeptide comprising at least 70% amino acid sequence identity to a polypeptide selected from the group consisting of SEQ ID NO: 2, SEQ ID NO: 4 and SEQ ID NO: 6.

In one embodiment the DNA mycovirus encodes at least two of the polypeptides, preferably all three of the polypeptides.

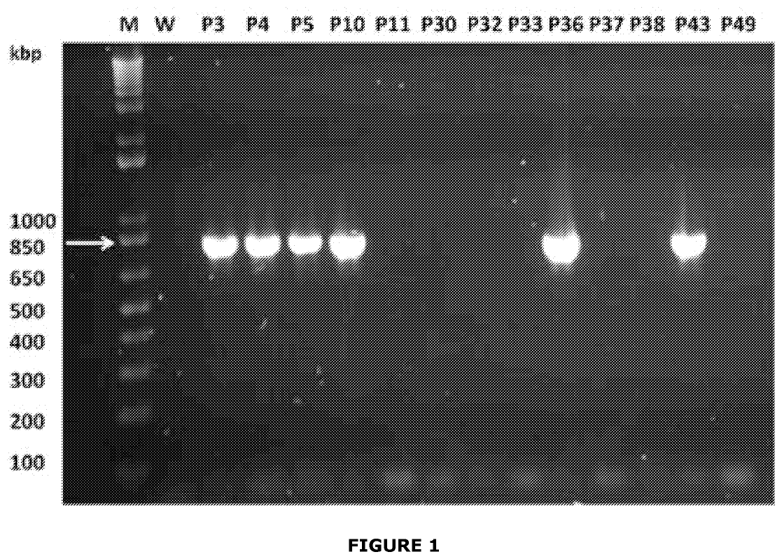

In one embodiment the DNA mycovirus encodes a polypeptide comprising at least 70%, preferably at least 75%, preferably at least 80%, preferably at least 85%, preferably at least 90%, preferably at least 95%, preferably at least 99% amino acid sequence identity to SEQ ID NO: 2, and at least one RCR or S3 helicase amino acid motif as shown in FIG. 1E. In one embodiment the polypeptide comprises at least two, preferably at least 3, preferably at least 4, preferably at least 5, preferably at least 6, preferably all 7 of the RCR and/or S3 helicase motifs shown in FIG. 1E. In one embodiment the RCR motifs are selected from the group consisting of motif I, motif II, GRS and motif III as shown in FIG. 1E. In one embodiment the S3 helicase motifs are selected from the group consisting of Walker-A, Walker-B and motif C as shown in FIG. 1E.

In one embodiment each motif in FIG. 1E consists essentially of the following amino acid residues:

Motif I-XLTXXX,
Motif II-XHXHXX,
Motif GRS-XXFDXXXXHPNXXXXX,
Motif III-YXXK,
the Walker-A-GXXXXGKT,
the Walker-B-XXDDX,
and
Motif C-of NXXX,

wherein X is any amino acid residue.

In one embodiment the RCR motifs consist essentially of, or consist of, Motif I (MLTYAQ), Motif II (HIHAY), GRS (DELDYCNHHPNILPIR) and Motif III (YVGK).

In one embodiment the S3 helicase amino acid motifs consist essentially of, or consist of SF3 Helicase Walker-A (GDTRLGKT), Walker-B (IFDDI) and Motif C (NTDP).

In one embodiment the DNA mycovirus encodes a polypeptide comprising Motif I (MLTYAQ), Motif II (HIHAY), GRS (DELDYCNHHPNILPIR), Motif III (YVGK), Walker-A (GDTRLGKT), Walker-B (IFDDI) and Motif C (NTDP).

In one embodiment the at least one polypeptide comprises at least 75%, preferably at least 80%, preferably at least 85%, preferably at least 90%, preferably at least 95%, preferably at least 99%, preferably 100% sequence identity to SEQ ID NO: 2, SEQ ID NO: 4 or SEQ ID NO: 6.

In one embodiment the at least two polypeptides comprise at least 75%, preferably at least 80%, preferably at least 85%, preferably at least 90%, preferably at least 95%, preferably at least 99%, preferably 100% sequence identity to two of SEQ ID NO: 2, SEQ ID NO: 4 and SEQ ID NO: 6.

In one embodiment each of the three polypeptides comprises at least 75%, preferably at least 80%, preferably at least 85%, preferably at least 90%, preferably at least 95%, preferably at least 99%, preferably 100% sequence identity each of SEQ ID NO: 2, SEQ ID NO: 4 and SEQ ID NO: 6 respectively.

In another aspect the invention relates to an isolated polypeptide comprising at least 70% sequence identity to an amino acid sequence selected from the group consisting of SEQ ID NO: 2, SEQ ID NO: 4 and SEQ ID NO: 6.

In one embodiment the isolated polypeptide comprises at least 70%, preferably at least 75%, preferably at least 80%, preferably at least 85%, preferably at least 90%, preferably at least 95%, preferably at least 99% amino acid sequence identity to SEQ ID NO: 2, and at least one RCR or S3 helicase amino acid motif as shown in FIG. 1E. In one embodiment the isolated polypeptide comprises at least two, preferably at least 3, preferably at least 4, preferably at least 5, preferably at least 6, preferably all 7 of the RCR and/or S3 helicase motifs shown in FIG. 1E. In one embodiment the RCR motifs are selected from the group consisting of motif I, motif II, GRS and motif III as shown in FIG. 1E. In one embodiment the S3 helicase motifs are selected from the group consisting of Walker-A, Walker-B and motif C as shown in FIG. 1E.

In one embodiment each motif in FIG. 1E consists essentially of the following amino acid residues:

Motif I-XLTXXX,
Motif II-XHXHXX,
Motif GRS-XXFDXXXXHPNXXXXX,
Motif III-YXXK,
the Walker-A-GXXXXGKT,
the Walker-B-XXDDX,
and
Motif C-of NXXX,

wherein X is any amino acid residue.

In one embodiment the RCR motifs consist essentially of, or consist of, Motif I (MLTYAQ), Motif II (HIHAY), GRS (DELDYCNHHPNILPIR) and Motif III (YVGK).

In one embodiment the S3 helicase amino acid motifs consist essentially of, or consist of SF3 Helicase Walker-A (GDTRLGKT), Walker-B (IFDDI) and Motif C (NTDP).

In one embodiment isolated polypeptide comprises Motif I (MLTYAQ), Motif II (HIHAY), GRS (DELDYCNHHPNILPIR), Motif III (YVGK), Walker-A (GDTRLGKT), Walker-B (IFDDI) and Motif C (NTDP).

In one embodiment the isolated polypeptide comprises at least 75%, preferably at least 80%, preferably at least 85%, preferably at least 90%, preferably at least 95%, preferably at least 99%, preferably 100% sequence identity to the amino acid sequence selected from the group consisting of SEQ ID NO: 2, SEQ ID NO: 4 and SEQ ID NO: 6.

In one embodiment the isolated polypeptide is a functional variant, analogue or derivative of a polypeptide comprising SEQ ID NO: 2, SEQ ID NO: 4 or SEQ ID NO: 6.

In another aspect the invention relates to an isolated nucleic acid sequence encoding a polypeptide of the invention.

In one embodiment the isolated nucleic acid sequence comprises at least 70% sequence identity to a nucleic acid sequence selected from the group consisting of SEQ ID NO: 3, SEQ ID NO: 5 and SEQ ID NO: 7. In one embodiment the isolated nucleic acid sequence comprises at least 75%, preferably at least 80%, preferably at least 85%, preferably at least 90%, preferably at least 95%, preferably at least 99%, preferably 100% sequence identity to SEQ ID NO: 3, SEQ ID NO: 5 or SEQ ID NO: 7.

In one embodiment the isolated nucleic acid sequence is selected from the group consisting of SEQ ID NO: 3, SEQ ID NO: 5 and SEQ ID NO: 7, or a degenerate sequence thereof.

In another aspect the invention relates to an isolated nucleic acid sequence comprising at least 70% sequence identity to SEQ ID NO: 1. In one embodiment the isolated nucleic acid sequence comprises at least 75%, preferably at least 80%, preferably at least 85%, preferably at least 90%, preferably at least 95%, preferably at least 99%, preferably 100% sequence identity to SEQ ID NO: 1.

In another aspect the invention relates to an isolated DNA mycovirus comprising SEQ ID NO: 1 or a degenerate strain thereof. In one embodiment the DNA mycovirus consists essentially of SEQ ID NO: 1. In one embodiment the DNA mycovirus consists of SEQ ID NO: 1. In one embodiment the DNA mycovirus is BGDaV1.

In another aspect the invention relates to a vector comprising a nucleic acid sequence according to the invention. In one embodiment the vector is selected from the group consisting of plasmids, bacteriophage, phagemids, cosmids, fosmids, bacterial artificial chromosomes, yeast artificial chromosomes, and phage artificial chromosomes.

In another aspect the invention relates to an isolated host cell comprising an isolated nucleic acid sequence, vector, polypeptide or DNA mycovirus or degenerate strain thereof of the invention.

In one embodiment the isolated host cell is a bacterial cell or a fungal cell, preferably a fungal cell. In one embodiment the fungal cell is a Botrytis spp. cell, preferably a B. cinerea, B. pseudocinerea, B. allii, B. paeoniae, B. porri, or B. tulipae cell.

In another aspect the invention relates to a hypovirulent fungal strain or part thereof comprising an isolated nucleic acid sequence, vector, polypeptide or DNA mycovirus or degenerate strain thereof of the invention. In one embodiment the isolated strain is a Botrytis spp., preferably B. cinerea, B. pseudocinerea, B. allii, B. paeoniae, B. porri, or B. tulipae cell.

In another aspect the invention relates to a composition comprising an isolated nucleic acid sequence, vector, polypeptide, DNA mycovirus or degenerate strain thereof, isolated host cell, hypovirulent fungal strain or part thereof, or a combination thereof, of the invention, and a carrier, diluent or excipient.

In some embodiments, the composition of the invention may comprise or consist essentially of a nucleic acid sequence, polypeptide, DNA mycovirus or degenerate strain thereof, isolated host cell, hypovirulent fungal strain or part thereof, or a combination thereof, as described herein for any other aspect of the invention.

In one embodiment the carrier is an agriculturally acceptable carrier, preferably water.

In some embodiments the composition comprises a DNA mycovirus or degenerate strain thereof, isolated host cell or hypovirulent fungal strain or part thereof according to the invention. In such embodiments the concentration of virus like particles (VLPs) of the DNA mycovirus, or the concentrations of cells and/or of hyphae or parts thereof of either the isolated host cells or hyphae of the hypovirulent fungal strain in the composition will depend on the utility to which the composition is put. Optimizing the concentration of VLPs, cells, and/or hyphae and/or parts thereof for a particular application is believed to be within the skill in the art.

In one embodiment the cells in a composition of the invention are viable cells.

In one embodiment the composition comprises hyphae or parts thereof of the hypovirulent fungal strain. In one embodiment the composition consists essentially of hyphae or parts thereof of the hypovirulent fungal strain.

In some embodiments the concentration of VLPs or cells in a composition of the invention ranges from about 1×10³ to about 1×10¹⁴, preferably about 1×10⁵ to about 1×10¹¹, preferably about 1×10⁶ to about 1×10⁹, preferably about 1×10⁷ to about 1×10⁸, preferably about 2×10⁷ PFU or CFU, preferably about 1×10⁷ PFU or CFU per gram for solid compositions, and per millilitre for liquid compositions.

In some embodiments the concentration of VLPs or cells in a composition of the invention ranges from 1×10³ to about 1×10¹⁴, preferably 1×10⁵ to about 1×10¹¹, preferably from 1×10⁶ to about 1×10⁹, preferably 1×10⁷ to about 1×10⁸, preferably 2×10⁷ CFU, preferably about 1×10⁷ CFU per gram for solid compositions, and per millilitre for liquid compositions.

In some embodiments the concentration of VLPs or cells in a composition of the invention ranges from about 1×10³ to 1×10¹⁴, preferably about 1×10⁵ to 1×10¹¹, preferably about 1×10⁶ to 1×10⁹, preferably about 1×10⁷ to 1×10⁸, preferably about 2×10⁷ CFU, preferably about 1×10⁷ CFU per gram for solid compositions, and per millilitre for liquid compositions.

In some embodiments the concentration of VLPs or cells in a composition of the invention ranges from 1×10³ to 1×10¹⁴, preferably 1×10⁵ to 1×10¹¹, preferably 1×10⁶ to 1×10⁹, preferably 1×10⁷ to 1×10⁸, preferably 2×10⁷ CFU, preferably about 1×10⁷ CFU, per gram for solid compositions, and per millilitre for liquid compositions.

Concentrations of VLPs or cells or hyphae or parts thereof that are effective as a biological control agent in the composition of the invention may vary depending on the form the VLP or cell is used in, physiological condition of the plant to which the VLP or cell is applied; type, concentration and degree of pathogen infection; temperature;

season; humidity; soil type; stage in the growing season; age of the plant; number and type of conventional pesticides and fungicides being applied and plant treatments (such as pruning, but not limited thereto). All factors may be taken into account in formulating a composition of the invention or in a composition for use in a method of the invention.

Compositions of the invention may be prepared using standard techniques known in the art and as described in the examples herein. In one embodiment the hyphae or parts thereof in the composition are prepared by macerating the hyphae and/or mycelia of a hypovirulent fungal strain as described herein, preferably a hypovirulent Botrytis spp. strain as described herein.

In one embodiment the composition comprises an agriculturally acceptable adjuvant. In one embodiment the agriculturally acceptable adjuvant is selected from the group consisting of an additional active agent and a formulation agent.

In one embodiment the agriculturally acceptable adjuvant is one or more additional active agents. In one embodiment the agriculturally acceptable adjuvant is one or more formulation agents.

In one embodiment the composition comprises a combination of one or more additional active agents and one or more formulation agents. In some embodiments the composition is formulated as pre-prepared composition or in a concentrated form. In some embodiments the composition comprises a solid or a liquid formulation.

In one embodiment composition of the invention comprises one or more agriculturally acceptable adjuvants. In one embodiment the agriculturally acceptable adjuvants are selected from the group of additional active agents and formulation agents. Preferably the one or more agriculturally acceptable adjuvant is an additional active agent. Preferably the one or more agriculturally acceptable adjuvant is a formulation agent.

In one embodiment the composition of the invention comprises a combination of one or more additional active agents and one or more formulation agents.

In some cases it may also be desirable to include one or more additional active agents in the compositions of the invention where such additional active agents are capable of contributing to the control (e.g., treatment and/or prevention) of plant pathogenic fungi including Botrytis spp., but not limited thereto.

Suitable additional active agents for use in the present invention may be capable of controlling plant pathogenic fungi including Botrytis spp. (but not limited thereto), or may be capable of potentiating the biocontrol effect of a DNA mycovirus, hypovirulent fungal strain, or composition of the invention for controlling Botrytis spp., particularly Botrytis cinerea. Additional active agents may be included directly in the composition of or useful in the invention, or may be applied separately, either simultaneously or sequentially as appropriate according to a method of the invention.

Suitable additional active agents include, but are not limited to plant defence elicitors including acibenzolar-S-methyl (Actigard/Bion, Syngenta), Azelaic acid, Pipecolinic acid, Jasmonic acid, Seaweed Mix, Lema oil, Foodcoat (DOMCA), Fungicover (bioDURACAL agricultura) and Ibuprofen, antagonistic microorganisms, potassium silicate, inorganic salts including calcium, potassium or sodium salts, stimulating agents including uronic acids, amnnans, and β 1-3 glucans, antibiotics, and other antibacterial and antifungal compounds including small organic and inorganic molecules.

In one embodiment the composition of the invention comprises one or more formulation agents.

In one embodiment the composition of the invention comprises a combination of one or more additional active agents and one or more formulation agents.

In one embodiment, the composition of the invention is formulated as a solid or a liquid formulation.

In one embodiment the composition of the invention may comprise one or more solid or liquid formulation agents. Any suitable formulation agent(s) may be used as known in the art. It is believed that the selection of a suitable formulation agent is within the skill of those in the art. For example, a suitable formulation agent may be a compound or other material that facilitates or optimizes the production, handling, storage, transport, application and/or persistence of the composition of, or for use in the invention on plants or on parts thereof, but not limited thereto.

Formulation agents can be specifically adapted for particular uses such as, but not limited to, preservation and maintenance of the biological control activity of the yeasts comprised in the composition of or for use in the invention during transportation from production facilities, storage on site, or during preparation of a final treatment mixture. Formulation agents may also be used for other purposes such as facilitating adhesion and persistence on plants or penetration into plant tissues, but not limited thereto. A suitable formulation may be solid, liquid, alone or in combination. Particularly suitable formulation agents include surfactants, dispersants, preservatives, wetting agents, emulsifiers, humectants, stickers, spreaders, stabilizers, penetrants, adhesion agents, pH buffers, and nutrients, either alone or in various combinations as may be determined by the skilled worker.

The composition of the invention may be provided as a pre-prepared composition ready for use, or in a concentrated, solid or liquid form.

In one embodiment, the composition is a pre-prepared composition having a solid or liquid formulation. In one embodiment the pre-prepared composition is a solid formulation selected from powders, pellets, granules and prills. In one embodiment the pre-prepared composition is a liquid formulation.

The composition of or for use in the invention may be provided in a pre-prepared form, or in a concentrated form. If provided in a dry form, the pre-prepared composition may be provided as a powder, granule, pellet or prill, but not limited thereto. In the case of a dry form, the composition is preferably in dehydrated, dried and/or encapsulated form. In some embodiments, the dehydrated, dried and/or encapsulated forms include additional protective agents as known in the art; e.g., lyoprotectants and the like.

In one embodiment, the composition may be provided in granule form. For example, a DNA mycovirus, cell or hypovirulent fungal strain or part thereof according to the invention may be provided in a granule having at least 0.5×10¹⁰ PFU/gm or CFU/gm, preferably 1×10¹⁰ PFU/gm or CFU/gm, preferably 2×10¹⁰ PFU/gm or CFU/gm. Where the pre-prepared composition is provided in a liquid form, particularly an aqueous form the composition may be provided as a dispersion, a suspension, a slurry, a cream, a paste or a gel, but not limited thereto. Preferably the pre-prepared form is provided as an aqueous liquid form that is suitable for and/or is adapted for spraying. In one embodiment a pre-prepared liquid form can be used per se for example as a dip to inoculate flowers, fruits, vegetables, seeds or plants, including plant cuttings.

In one embodiment a pre-prepared composition of the invention is formulated for use on plants, particularly grape vines. For example, the VLPs, cells or hypovirulent fungal strain or part thereof according to the invention can be mixed with an agriculturally acceptable carrier liquid that enables spray applications, a fertilizer, an elicitor, an adjuvant, a wetting agent, or any other suitable additional agent as required. In the pre-prepared composition for use according to the methods of the invention, the VLPs, cells or hypovirulent fungal strain or part thereof may also be mixed with an agriculturally acceptable carrier liquid that enables spray applications, a fertilizer, an elicitor, an adjuvant, a wetting agent, or any other suitable additional agent as required.

The formulation of a DNA mycovirus, cell or hypovirulent fungal strain or part thereof according to the invention into a pre-prepared composition of the invention and the final form of the pre-prepared composition for application to the plant or part thereof is believed to be within the skill in the art. For example, the final form of the composition is formulated with an agriculturally acceptable carrier such as water to form a spray, foam, drench, injectable, gel, dip or paste, but not limited thereto. In one embodiment, a composition of the invention may be applied to plants or parts thereof by spraying, dipping, painting, spreading, coating, rubbing or brushing, or a combination thereof. Preferably the composition is formulated as an aqueous suspension or dispersion for spray or mist application. In one embodiment the spray or mist application is to grape vines, cherry trees and/or fruit and/or vegetables and/or flowers.

In one embodiment the composition of the invention is in concentrated form. In one embodiment the concentrated form is a solid form selected from cakes, powders, granules, pellets and prills. In one embodiment the concentrated form is a liquid formulation. In one embodiment the liquid formulation is an emulsion or gel.

Where the composition of the invention is provided in a concentrated form it may require additional formulation by the user to produce a composition ready for application to a plant or part thereof. For example, the concentrated form can be mixed with various formulation agents to form a final composition for plant application. A preferred formulation is agent is water or an aqueous solution in which an appropriate amount of the concentrated from of the composition is dissolved (e.g., granules or powders) or diluted (e.g., liquid suspensions or dispersions) to obtain a final composition for application to a plant.

If the DNA mycovirus, cells or hypovirulent fungal strain or part thereof according to the invention is dehydrated in the concentrated form then rehydration as known in the art will be required if the composition for application to the plant is intended to be in liquid form. Rehydration may be carried out using customary precautions for rehydrating the yeast as known in the art; for example rehydration may be achieved advantageously at temperatures between 20 and 25° C., but not limited thereto.

### Methods—Botrytis spp.

In another aspect the invention relates to a method of reducing the virulence of at least one phytopathogenic fungus comprising contacting the fungus with an isolated DNA mycovirus or degenerate strain thereof of the invention.

In one embodiment the at least one phytopathogenic fungus a Botrytis spp., preferably B. cinerea, B. pseudocinerea, B. allii, B. paeoniae, B. porri, or B. tulipae.

In another aspect the invention relates to a method of Botrytis spp. biocontrol comprising contacting at least one Botrytis spp. with an isolated DNA mycovirus, or degenerate strain thereof.

In one embodiment the DNA mycovirus or degenerate strain thereof is a DNA mycovirus or degenerate strain thereof according to any other aspect of the invention. In one embodiment the DNA mycovirus or degenerate strain thereof is comprised in a composition as described herein for any other aspect of the invention. In one embodiment the composition consists essentially of the DNA mycovirus or degenerate strain thereof.

In one embodiment contacting is to or on a plant or part thereof.

In one embodiment the plant or part thereof is selected from the group of monocotyledonous plants, dicotyledonous plants, annual, biannual and perennial plants, native New Zealand plants, vegetable plants or harvested vegetables, fruit plants or trees or harvested fruits, flower bearing plants or trees or harvested flowers, cereal plants, oleaginous plants, proteinous plants, ligneous plants, and ornamental plants.

In one embodiment, a plant or part thereof is an agriculturally important crop plant, cultivar or product thereof selected from corn plants, tobacco plants, wheat plants, sugar cane plants, rapeseed plants, barley plants, rice plants, sorghum plants, millet plants, soya bean plants, lettuce plants, cabbage plants, onion plants, garlic plants, and canola plants.

In one embodiment the plant or part thereof is an agriculturally important plant, cultivar thereof, or product thereof selected from the group consisting of agriculturally important vines and agriculturally important fruit trees, flower-producing plants, and cultivars and products thereof. In some embodiments the flower producing plants are peonies or tulips. In some embodiments the agriculturally important fruit trees or cultivars thereof are selected from grapevines, olive trees, apple trees, pear trees, citrus fruit trees, banana palms, pineapple plants, peach trees, apricot trees, cherry trees, walnut trees, hazelnut trees, strawberry plants, blueberry plants, raspberry plants, blackberry plants, and the products thereof are grapes, olives, apples, pears, citrus fruits, bananas, pineapples, peaches, apricots, cherries, walnuts, hazelnuts, strawberries, blueberries, raspberries, blackberries Preferably the agriculturally important vines or cultivars thereof are selected from potato vines, beetroot vines, bean vines, pea vines, tomato vines, cucumber vines, melon vines, berry vines, grape vines and kiwifruit vines and the products thereof are potatoes, beetroots, beans, peas, tomatoes, cucumbers, melons, berries, grapes and kiwifruits respectively. Preferably the agriculturally important vine is a grapevine or grapevine scion or a cultivar thereof, and the product is a grape.

In one embodiment the grape vine or grape vine scion is a Vinus spp., or a cultivar thereof, preferably a V. vinifera, or cultivar thereof. In some embodiments the V. vinifera is a wine grape variety, preferably Sauvignon blanc, Pinot Gris, Chardonnay, Riesling, Merlot, Syrah or Shiraz, Cabernet sauvignon, Cabernet franc, Tempranillo, or Grenache. In some embodiments the V. vinifera is an eating grape variety, preferably “Thompson Seedless”, Flame Seedless, Red globe, Concord, Cardinal, Ruby Roman, Delaware, or Canadice variety. In some embodiments the Vinus spp. is a grafted grapevine having a root stock that is not Vinus vinifera.

In one embodiment the strawberry plant is a Pajaro or Camarosa cultivar.

In one embodiment the part thereof is a flower or part thereof or a fruit or part thereof.

In one embodiment, the plant or part thereof is a flower-bearing plant. In one embodiment the flower-bearing plant is a perennial flowering plant. In one embodiment the perennial flower bearing plant is in the family Primulaceae, preferably the subfamily Myrsinoideae, preferably a Cyclamen spp., preferably C. persicum.

In one embodiment, contacting comprises applying the DNA mycovirus or degenerate strain thereof or a composition comprising the DNA mycovirus or a degenerate strain thereof to the plant or part thereof by applying to or within the seeds, leaves, stems, flowers, fruits, trunks and/or roots of the plant or part thereof. Preferably application is by spraying, misting, dipping, dripping, dusting, painting, spreading, injecting or sprinkling. In some embodiments contacting comprises disrupting the plant cuticle, when present, to allow the DNA mycovirus or a degenerate strain thereof to come into contact with the cells or intercellular spaces of the plant or part thereof. Applications can be made once only, or repeatedly as required. Also contemplated herein is application at various times of year and/or during various stages of the plant life cycle, as determined appropriate by the skilled worker.

The DNA mycovirus or degenerate strain thereof, or a composition comprising the DNA mycovirus or degenerate strain thereof may be applied at the appropriate time during the year and at the appropriate stage of plant development as may be determined by a skilled worker. For example the DNA mycovirus or degenerate strain thereof, or a composition comprising the DNA mycovirus or degenerate strain thereof may be applied from bud-burst to flowering, during flowering and post flowering/fruit set period but not limited thereto.

In one embodiment, applying is by spraying onto stems and/or shoots and/or leaf surfaces and/or onto flowers and/or onto fruit and/or onto vegetables.

In one embodiment, applying to the roots is by ground spraying, mechanical incorporation or by mixing with enriching agents or fertilizers prior to application in the usual way.

In another aspect the invention relates to a method of treating at least one plant disease caused by a phytopathogenic fungus comprising contacting the plant with an isolated DNA mycovirus or degenerate strain thereof of the invention or a hypovirulent fungal strain or part thereof of the invention, or both.

In another aspect the invention relates to a method of controlling at least one phytopathogenic fungus comprising contacting the fungus with an isolated DNA mycovirus or degenerate strain thereof of the invention or a hypovirulent fungal strain or part thereof of the invention, or both.

In some embodiments, the isolated DNA mycovirus or degenerate strain thereof is as described herein for any other aspect of the invention. In some embodiments the isolated hypovirulent fungal strain or part thereof is as described herein for any other aspect of the invention. In some embodiments the isolated DNA mycovirus or degenerate strain thereof, or isolated hypovirulent fungal strain or part thereof are comprised in composition as described herein. In some embodiments the composition consists essentially of the isolated DNA mycovirus or degenerate strain thereof, or isolated hypovirulent fungal strain or part thereof. In some embodiments contacting is as described herein for any other aspect of the invention. In some embodiments the phytopathogenic fungus is as described herein for any other aspect of the invention. In some embodiments the plant or part thereof is as described herein for any other aspect of the invention.

In another aspect the invention relates to an isolated DNA mycovirus or degenerate strain thereof of the invention for use in controlling at least one phytopathogenic fungal strain. In some embodiments, the isolated DNA mycovirus or degenerate strain thereof is as described herein, is provided as described herein, is comprised in a composition as described herein and/or is used as described herein for any other aspect of the invention. In some embodiments the phytopathogenic fungal strain is as described herein for any other aspect of the invention.

In another aspect the invention relates to an isolated hypovirulent fungal strain or part thereof of the invention for use in controlling at least one phytopathogenic fungal strain. In some embodiments the isolated hypovirulent fungal strain or part thereof is as described herein, is provided as described herein, is comprised in a composition as described herein, and/or is used as described herein for any other aspect of the invention. In some embodiments the phytopathogenic fungal strain is as described herein for any other aspect of the invention.

In another aspect the invention relates to an isolated DNA mycovirus, or a degenerate strain thereof, for use in controlling Botrytis spp. fungi. In some embodiments, the isolated DNA mycovirus or degenerate strain thereof is as described herein, is provided as described herein, is comprised in a composition as described herein, and/or is used as described herein for any other aspect of the invention. In some embodiments the Botrytis spp. fungi are as described herein for any other aspect of the invention.

In another aspect the invention relates to an isolated hypovirulent Botrytis spp. fungus or part thereof for use in controlling Botrytis spp. fungi. In some embodiments, the isolated Botrytis spp. fungus or part thereof is as described herein, is provided as described herein, is comprised in a composition as described herein, and/or is used as described herein for any other aspect of the invention. In some embodiments the Botrytis spp. fungi are as described herein for any other aspect of the invention.

Various aspects of the invention will now be illustrated in non-limiting ways by reference to the following examples.

### EXAMPLES

### Example 1—Fungal Isolates and Culturing Conditions

Five hundred isolates of B. cinerea were obtained from Landcare Research, New Zealand (Table 1). These isolates were chosen on the basis that they were isolated from asymptomatic plants; thereby increasing the chances of finding hypovirulent isolates and/or circular DNA mycoviruses. Isolates were subcultured and maintained on potato dextrose agar (PDA) at 4° C. until used.

TABLE 1
Identification number of
isolates of Botrytis cinerea
from Landcare Research.

Isolate identification
number
339-2
339-3
339-4
339-6
339-7
339-8
339-10
339-11
339-12
339-13
339-14
339-15
339-17
339-19
339-21
339-23
339-25
339-26
339-27
339-28
339-30
339-33
339-35
339-36
339-37
339-38
339-40
339-43
339-44
339-47
339-48
339-49
339-50
339-51
339-52
339-53
339-54
339-57
339-58
339-59
339-60
339-61
339-62
339-63
339-64
339-66
339-67
339-68
339-69
339-71
339-75
339-76
339-77
339-78
339-79
339-80
339-81
339-82
339-83
339-84
339-85
339-86
339-87
339-88
339-89
339-90
339-91
339-92
339-93
339-94
339-95
339-96
339-98
339-99
339-100
339-101
339-103
339-104
339-105
339-106
339-108
339-109
339-110
339-111
339-112
339-113
339-114
339-115
339-116
339-117
339-118
339-119
327-1
327-3
327-4
327-5
327-6
327-7
327-8
327-9
327-10
327-12
327-14
327-15
327-16
327-18
327-20
327-22
327-23
327-24
327-25
327-26
327-27
327-28
327-29
327-30
327-54
327-55
327-56
327-57
327-58
327-61
327-62
327-63
327-64
327-65
327-66
327-68
327-70
327-71
327-72
327-73
327-76
327-77
327-80
327-83
327-84
327-85
327-86
327-88
327-89
327-94
327-97
327-98
327-100
327-101
327-102
327-104
327-105
327-107
327-108
327-111
327-114
327-115
327-119
327-121
327-122
327-123
327-129
327-131
327-133
327-134
327-143
327-160
334-201
334-202
334-203
334-204
334-205
334-206
334-207
334-208
334-209
334-210
334-211
334-212
334-213
334-214
334-215
334-217
334-218
334-219
334-220
334-221
334-222
334-223
334-224
334-225
334-226
334-229
334-230
334-231
334-232
334-233
334-234
334-235
334-236
334-237
334-238
334-239
334-240
334-241
334-243
334-244
334-245
334-246
334-247
334-248
334-249
334-251
334-252
334-253
334-254
334-255
334-256
334-257
334-258
334-259
334-261
334-262
334-263
334-265
334-266
334-267
334-268
334-269
334-270
334-271
334-273
334-274
334-276
334-277
334-279
334-282
334-283
334-284
334-287
334-288
334-289
334-291
334-292
334-293
334-295
334-296
334-297
334-299
334-300
334-301
334-303
334-304
334-305
334-306
334-307
334-310
334-311
334-312
334-314
334-315
334-316
340-1
340-2
340-3
340-4
340-5
340-6
340-7
340-8
340-9
340-10
340-11
340-12
340-13
340-15
340-16
340-17
340-18
340-19
340-20
340-22
340-23
340-24
340-25
340-26
340-27
340-28
340-29
340-31
340-32
340-33
340-34
340-35
340-36
340-37
340-38
340-40
340-41
340-43
340-44
340-45
340-46
340-47
340-48
340-49
340-50
340-51
340-52
340-53
340-54
340-55
340-56
340-57
340-58
340-59
340-60
340-61
340-62
340-64
340-65
340-66
340-67
340-68
340-69
340-70
340-71
340-72
340-73
340-74
340-75
340-76
340-77
340-78
340-79
340-80
340-81
340-83
340-84
340-85
340-86
340-87
340-88
340-89
340-90
340-91
340-92
340-93
340-94
340-95
340-96
340-97
340-98
340-99
340-100
340-101
340-102
340-103
340-104
340-105
340-106
340-107
340-108
340-109
340-110
340-111
340-112
340-113
340-114
340-115
340-116
340-117
340-118
340-119
340-120
331-201
331-202
331-203
331-204
331-205
331-206
331-207
331-208
331-209
331-210
331-211
331-212
331-213
331-214
331-215
331-216
331-217
331-218
331-219
331-220
331-221
331-222
331-223
331-224
331-225
331-226
331-227
331-228
331-229
331-230
331-231
331-232
331-233
331-234
331-235
331-236
331-237
331-238
331-239
331-240
331-241
331-242
331-243
331-244
331-245
331-246
331-247
331-248
331-249
331-250
331-251
331-252
331-253
331-254
331-255
331-256
331-257
331-258
331-259
331-260
331-261
331-262
331-263
331-264
331-265
331-266
331-267
331-268
331-269
331-270
331-271
331-272
331-273
331-274
331-275
331-276
331-277
331-278
331-279
331-280
331-281
331-282
331-283
331-284
331-285
331-286
331-287
331-288
331-289
331-290
331-291
331-292
331-293
331-294
331-295
331-296
331-297
331-298
331-299
331-300
331-301
331-302
331-303
331-304
331-305
331-306
331-307
331-308
331-309
331-310
331-311
331-312
331-313
331-314
331-315
331-316
332-201
332-202
332-203
332-204
332-205
332-206
332-207
332-208
332-209
332-210
332-211

Soil fungi (273 isolates) from different regions in New Zealand were isolated using serial dilution and pour plate techniques (Table 2). Soil fungi were maintained on malt extract agar (MEA) media at 4° C.

TABLE 2
Soil sample details

| Number of isolates | Sources of soil samples for analysis |
|---|---|
| 34 | Warkworth, New Zealand |
| 58 | Amberley, New Zealand |
| 53 | Rotorua, New Zealand |
| 24 | Porirua, New Zealand |
| 62 | Wanaka, New Zealand |
| 42 | Auckland, New Zealand |
| 273 | Total |

### Example 2—Viral Nucleic Acid Purification, Enrichment and Sequencing

Isolates of B. cinerea were cultured on cellophane covered Potato Dextrose Agar (PDA) and incubated at 20° C. for 5 days. Approximately 250 mg of each isolate mycelium was collected and mycelia combined in groups of ten prior to virus-like particle (VLP) partial purification and DNA extraction. This resulted in 50 samples (representing all of the 500 isolates) that were further processed and sequenced. Fungal mycelia were homogenised and mixed with 5 ml of SM (0.1 M NaCl, 50 mM Tris-HCl, pH 7.4) or phosphate buffer. Homogenates were clarified by centrifugation at 10,000×g for 5 min and supernatants were filtered through 0.45 μm syringe filters. Total viral nucleic acid was extracted from these filtrates using High Pure Viral Nucleic Acid Large Volume Kit (Roche, Switzerland) according to the manufacturer's protocol and enriched for circular DNA by rolling-circle amplification (RCA) using Illustra™ TempliPhi™ DNA Amplification Kit (GE Healthcare, USA) as described by the manufacturer. RCA products from the 50 samples were equimolar pooled before proceeding to sequencing using Illumina Hiseq2000 100 bp at Macrogen Inc. (Seoul, South Korea).

Soil fungi were cultured on cellophane covered MEA and incubated at room temperature for 5-7 days. To partially purify VLPs, 200 mg of each isolate mycelium was homogenised, mixed with 700 μl of SM buffer, homogenates clarified by centrifugation at 10,000×g for 5 min and then supernatants were filtered through 0.2 μm syringe filters. Total viral nucleic acid was extracted from 200 μl of the filtrates using High Pure Viral Nucleic Acid Kit (Roche) according to the manufacturer's protocol. Circular DNA elements were enriched by RCA using Illustra™ TempliPhi™ DNA Amplification Kit (GE Healthcare) as described by the manufacturer. RCA products were pooled and sent for sequencing using Illumina Hiseq2500 100 bp Paired-end at Macrogen Inc. (Seoul, South Korea).

### Bioinformatics and Illumina Sequencing Analysis

Illumina reads obtained from the first sequencing run (to detect and sequence circular DNA viruses from 500 isolates of B. cinerea) with quality scores of less than Q20 were filtered out using The Galaxy Project server (Goecks et al. 2010), and the remaining reads were trimmed to remove low-quality sequence stretches at the 5′ end as determined by the FastQC report (http://www.bioinformatics.babraham.ac.uk/projects/fastqc/). This was followed by assembling the reads into contigs using the de novo assembly tool of Geneious R8.1 (http://www.geneious.com, Kearse et al., 2012) set to medium sensitivity and default parameters. Consensus sequences of assembled contigs longer than 1 kb were used to identify circular virus-like sequences using BLASTX (Altschul et al. 1990) analysis against the non-redundant (nr) database of NCBI.

### Detection and Sequence Confirmation of BGDaV1

Two pairs of primers were designed for PCR detection and amplification of two overlapping segments that together cover the full-length sequence of the circular viral genome of BGDaV1 (tentative name Botrytis gemydayravirus) recovered by Illumina sequencing (first run) as shown later in the results section. The 50 presumed viral DNA pools (no RCA enrichment) were PCR screened for the presence of BGDaV1 sequence. DNA was purified from isolates of each pool that tested positive for BGDaV1 using a ZR Fungal/Bacterial DNA MiniPrep or a High Pure Viral Nucleic Acid Kit (Roche) and PCR-screened for BGDaV1.

TABLE 3
Primer names, sequences, and expected amplicon
sizes for PCR detection and amplification of
Botrytis gemydayravirus 1 (BGDaV1) DNA.

| Primer | | Amplicon | |
|---|---|---|---|
| name | Primer sequence | size (bp) | |
| P01-1F1 | GGAGATACAAGCCAAAGGGG | 830 | SEQ ID NO: 8 |
| P01-1R1 | CTGTTTGCGCCTCTTTGGGG | | SEQ ID NO: 9 |
| P01-1F2 | CTACTCTTCATTTGCTGCT | 1250 | SEQ ID NO: 10 |
| | GCC | | |
| P01-1R2 | CTTGCCCAACGACCTAGCCC | | SEQ ID NO: 11 |

### BGDaV1 Sequence and Phylogenetic Analyses

BGDaV1 circular sequence was assembled from Sanger-sequenced reads using Geneious R8.1 (http://www.geneious.com, Kearse et al. 2012). Multiple sequence alignment and detection of conserved motifs of the Rep of the BGDaV1 was carried out using MUSCLE (Edgar 2004). For phylogenetic analysis, Rep amino acid sequences of circular ssDNA viruses were aligned using MUSCLE plug-in of MEGA7 (Kumar et al. 2016). Aligned sequences were trimmed to ensure they were of the same length. The best-fit substitution model was detected and maximum likelihood (ML) phylogenetic tree constructed using MEGA7 software with bootstrapping of 100 replicates. The LG model combined with gamma-distributed rates across sites was used.

Circular Rep-Encoding ssDNA (CRESS) Recovery

Analysis of Illumina reads (from the first run) revealed the presence of a novel ssDNA-like sequence similar to that of dragonfly-associated circular virus 1 (DfasCV-1) (Rosario et al. 2012) and other circular plant and fungal DNA viruses. Six pools of DNA (originating from 60 B. cinerea isolates) were found to contain sequences similar to that detected by Illumina sequencing and closely related to DfasCV-1 (FIG. 1). These amplicons were collectively termed BGDaV1.

Testing the isolates representing the six virus-positive pools revealed that eleven isolates (339-13, 339-19, 339-30, 339-34, 339-38, 339-42, 339-48, 339-49, 339-98, 339-99 and 339-101) contained BGDaV1, all of which were isolated from the same vineyard (Matua Valley) in Auckland.

### BGDaV1 Sequence Analysis

The Sanger sequencing confirmed sequence of BcCDV-1 (FIG. 1D) is 1701 nt long with three unidirectional ORFs. The longest ORF (ORF I) is 966 nt long (nt position: 152-1117) whereas the remaining two ORFs, ORF II and III, are overlapping with lengths of 375 (nt position: 1137-1511) and 294 nt (nt position: 1454-46), respectively. The viral genome contains two intergenic regions; a long intergenic region (LIR) of 105 nt (nt position: 74-151) between ORF III and ORF I and a short intergenic region (SIR) of 19 nt (nt position: 1118-1136) between ORF I and II. A putative nonanucleotide sequence motif (CTATCAACAC) was identified at the top of a stem-loop structure located at the terminus of ORF III. ORF I codes for a 321 aa long protein with calculated molecular mass of 36.7 kDa. BLASTx search of its sequence revealed that it is closely related to Reps of circular viral-like sequences recovered from various environmental sources, insects, plants and the phytopathogenic fungus S. sclerotiorum.

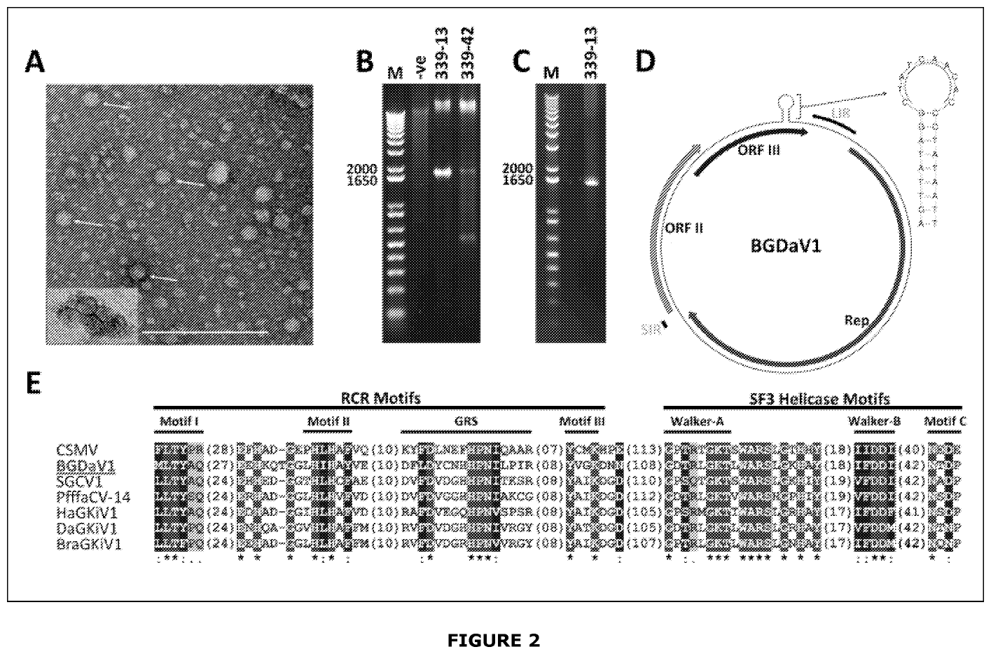

BGDaV1 Rep shared the highest aa sequence identity (39%) with that of DfasCV-1 (Rosario et al. 2012) whereas its closest assigned-to-known-host viruses were the mycovirus SsHADV-1 (accession number: YP_003104706; 35% identity) isolated in China and an Australian plant-infecting mastrevirus, chloris striate mosaic virus (CSMV; accession number: AFN80688; 32% identity). The Rep contained the conserved PCR (Motif I (MLTYAQ), Motif II (HIHAY), GRS (DELDYCNHHPNILPIR) and Motif III (YVGK)) and SF3 Helicase (Walker-A (GDTRLGKT), Walker-B (IFDDI) and Motif C (NTDP)) motifs described for BGDaV1-closely related ssDNA viruses (FIG. 2E).

### Phylogenetic Analysis

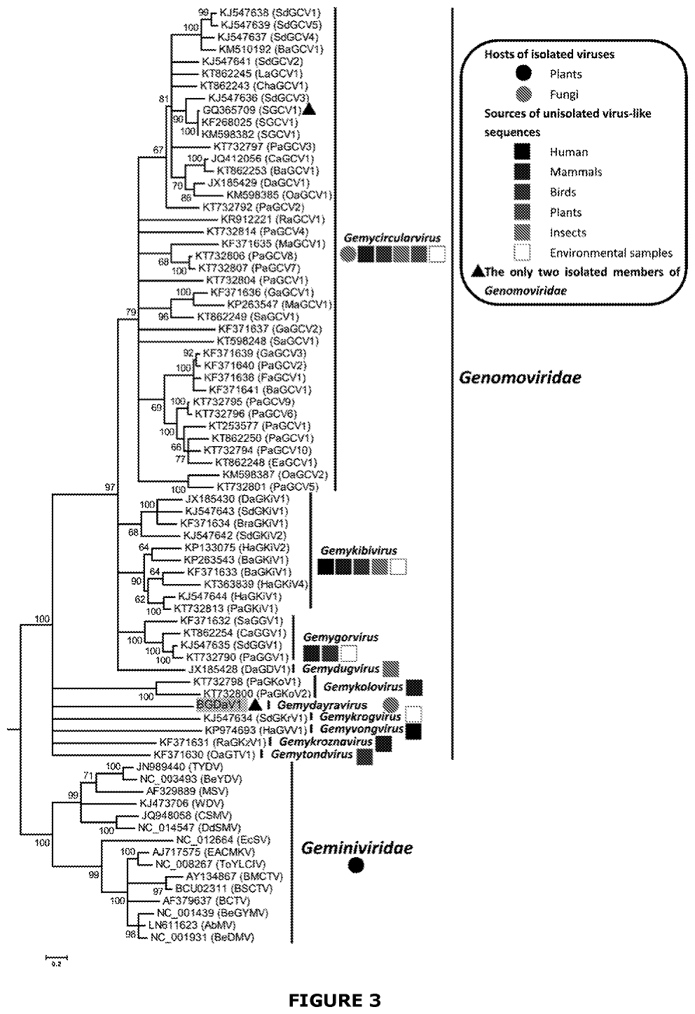

The ML tree based on the Rep sequence of BGDaV1 and other circular ssDNA sequences (FIG. 3) revealed that BGDaV1 is closely related, but distinct from, sequences in the genus Gemycircularvirus of the family Genomoviridae.

### Example 3 —Mechanical Transmission of BGDaV1 Particles

### BGDaV1 Particles Purification

A 10 gram portion of isolate 339-13 mycelia was ground to a fine powder in liquid nitrogen using sterilised mortar and pestle. The powder was transferred to a sterilised 50 ml falcon tube and a 20 ml aliquot of 0.1 M sodium phosphate buffer (pH 7) was added. The tube was shaken on ice for 10 mins, a 10 ml aliquot of chloroform was added and the tube was further shaken on ice for 30 mins prior to being centrifuged at 10000×g for 30 min at 4° C. The aqueous phase was separated between two ultracentrifuge tubes and the tubes were spun at 120000×g for 80 min. Following ultracentrifugation, the pellet was resuspended in a small volume of 0.02 M sodium phosphate buffer (pH 7), the suspension clarified by low speed centrifugation at 10000×g for 10 min at 4° C., the supernatant made up to 10 ml using 0.02 M sodium phosphate buffer (pH 7) and ultracentrifugation repeated as above. The resultant pellet was resuspended and clarified as above and the supernatant was examined by transmission electron microscope for the presence of virus particles.

### Virus-Like Particles (VLPs) Purification and TEM

VLPs from isolate 339-13 (tested+ve for BGDaV1) were purified and characterized as isometric VLPs (˜22 nm in diameter; FIG. 2A). Attempts to co-purify the viral DNA along with the fungal host genome from different isolates followed by detection of the viral DNA by agarose gel electrophoresis were unsuccessful. Without wishing to be bound by theory the inventors believe that this result is likely due to the viral DNA being present in low concentration that are undetectable by agarose gel electrophoresis. It was possible to detect linear dsDNA form of BGDaV1 by using RCA to enrich for the viral DNA followed by RCA digestion of its genome using a single cutter restriction enzyme (FIG. 2B). This also revealed the presence of a defective form (truncated genome) of BGDaV1 (˜500 nt) in isolate 339-42 (FIG. 2C).

### Screening BGDaV1-Containing Isolates for the Presence of RNA Viruses

A dsRNA purification protocol, as described by Khalifa & Pearson (2014), was used to screen BGDaV1-containing isolates for the presence of RNA viruses. The purified dsRNAs were electrophoretically separated on a SYBR safe pre-stained 1% (w/v) agarose gel in 1×TAE buffer (pH 7.4), visualised and photographed under UV using a Gel Doc (Bio-Rad, CA, USA).

Presence of dsRNA in BGDaV1-Containing Isolates

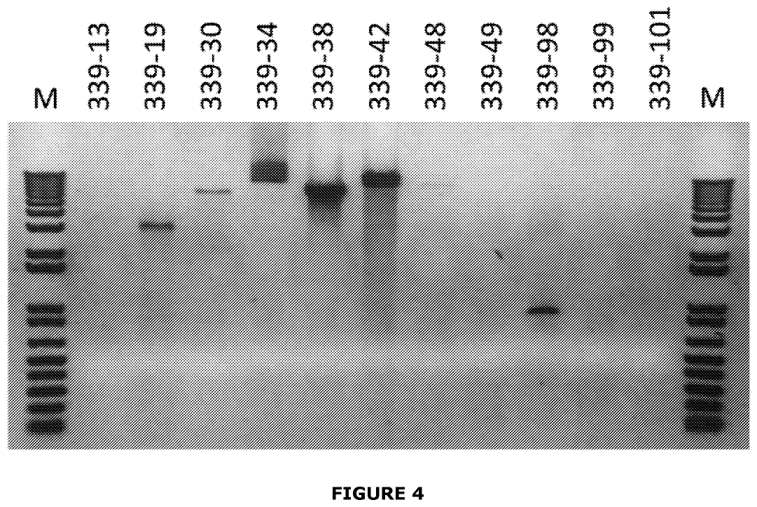

BGDaV1-containing isolates were tested for the presence of other RNA viruses using a dsRNA detection method. As shown in FIG. 4, dsRNAs were detected in seven isolates. Isolates 339-13, 339-49, 339-99 and 339-101 appeared to be dsRNA free and hence suitable for further transmission and pathogenicity experiments.

### Infectivity of BGDaV1 as Purified Particles

To study the mechanical transmissibility of BGDaV1 associated with three dsRNA-free isolates (339-13, 339-49 and 339-101), purified VLPs were applied to the growing margins of a virus-free B. cinerea isolate 702. After a 4 day incubation period at 20° C., mycelial plugs were transferred from the growing margins of the inoculated colonies onto new MEA plates to produce isolates 702-V13, 702-V49 and 702-V101. Total DNA was extracted from the resultant isolates and the transmission of BGDaV1 was tested by PCR using primers P01-1F1 and P01-1R1. The transmission experiment was repeated three times and PCR testing of BGDaV1 was performed on three successive subcultures of the new progeny.

### Transmissibility of BGDaV1

As shown in Table 4, BGDaV1 is mechanically transmissible as purified particles when applied on a virus-free isolate.

TABLE 4
Mechanical transmission and stability of BcGCV1. Presence (+) or absence (−)
of BcGCV1 in different sub-cultures of newly developed progeny as detected by PCR.

| | Experiment 1 | | | Experiment 2 | | | Experiment 3 | | |
|---|---|---|---|---|---|---|---|---|---|
| Virus donor | 339-13 | 339-49 | 339-101 | 339-13 | 339-49 | 339-101 | 339-13 | 339-49 | 339-101 |
| Sub-culture 1 | No transmission* | | | + | + | + | − | +¹ | +¹ |
| Sub-culture 2 | | | | − | + | + | − | +¹ | +¹ |
| Sub-culture 3 | | | | − | + | − | − | − | + |

*This was done on the first VLP purification attempt and negative transmission might have been due to failure in efficiently purifying the viruses
¹isolates used for virulence assessment.

Effect of BGDaV1 Infection on B. cinerea

To study the effect of BGDaV1 on the virulence of B. cinerea, mycelial plugs from cultures produced from the third transmission experiment were applied to detached canola leaves. Also, a mixture of VLPs purified from isolates 339-49 and 339-101 was applied directly to canola leaves resulting in prophylactic application of the two viruses and a mycelia plug of the virus-free isolate 702 was applied to the virus mixture on canola (Brassica napus) leaves. Inoculated leaves were incubated for 4-5 days before the diameter of the B. cinerea lesion on the leaf was measured. Each treatment was performed in triplicate.

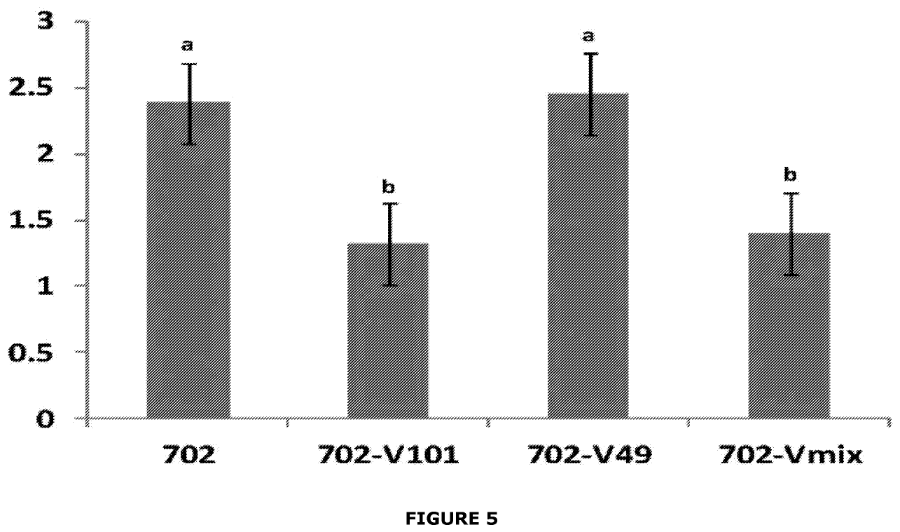

The lesion diameter of B. cinerea isolate 702-V101 or 702-Vmix is significantly less (P<0.050) than either B. cinerea 702 alone or B. cinerea 702-V49 (FIG. 5). The experiment was repeated for a second time using mycelia plugs from experiment 3 sub-culture 2 (Table 4). In this biological replicate there was no significant difference between the lesion diameters developed by virus-free and virus-infected isolates.

Further Effects of BGDaV1 Infection on B. cinerea

The inventors further investigated ability of BGDaV1 to replicate in and confer hypovirulence to Botrytis cinerea growing on grape berry, grape vine, kiwifruit, strawberry, and cyclamen.

### Methods and Materials

### Fungal Isolates and Virus Status Confirmation.

Fresh botrytis cultures (one virus-free and four virus-infected cultures) were sourced from Landcare Research (Table 5) and isolates were sub-cultured on PDA plates as described previously (Khalifa and MacDiarmid, 2017). To confirm virus status of cultures, total DNA was extracted from approximately 100 mg of mycelium from each isolate by either a conventional CTAB method or the Qiagen Plant total extraction kit, and tested by end-point PCR as described previously (Khalifa and MacDiarmid, 2017).

TABLE 5
Botrytis cultures used in this example

| Botrytis isolate name | | |
|---|---|---|
| Khalifa and | ICMP number | |
| MacDiarmid, 2017 | (this report) | Virus status |
| 702-01 | 14118 | Virus-free |
| 339-13 | 21918 | BGDaV1 positive |
| 339-49 | 21919 | BGDaV1 positive |
| 339-99 | 21920 | BGDaV1 positive |
| 339-101 | 21921 | BGDaV1 positive |

### Biological Assay

The biological assay to demonstrate BGDaV1 confers hypovirulence on Botrytis cinerea was performed twice on cyclamen and strawberry (two cultivars, Pajaro and Camarosa) leaves, and table grape berries, and once on Hort16A kiwifruit leaves. Each biological assay experiment tested six treatments (the five Botrytis isolates and a negative Botrytis control i.e. potato dextrose agar (PDA) with no inoculum) for each plant medium in triplicate.

To remove potential residual fungicide applications from the cyclamen and strawberries plants were rinsed under water and left for at least a week before leaves were used for experiments. Detached leaves and table grapes were surface sterilised (biological assay replicate 1 washed leaves in 80% ethanol for 3 to 5 min and then rinsed with autoclaved water; biological assay replicate 2 washed leaves in 10% bleach for 20 min and then rinsed with autoclaved water) and then placed on to water agar with the petiole lowered into the agar. One 4 mm plug (either a PDA plug with no mycelium or a mycelium plug from one of the Botrytis isolates) was placed on a detached leaf or table grape (plugs were either placed on table grapes with no incision or grapes with a small incision). Inoculated leaves and table grapes were incubated at room temperature for 4-5 days and photographs were taken. To investigate Botrytis penetration into the table grape, grapes were cut in half 7 days post inoculation (dpi) and photographs were taken.

### Results

All four BGDaV1 containing botrytis isolates and the virus-free isolate were successfully re-initiated from new samples purchased from Manaaki Whenua Landcare.

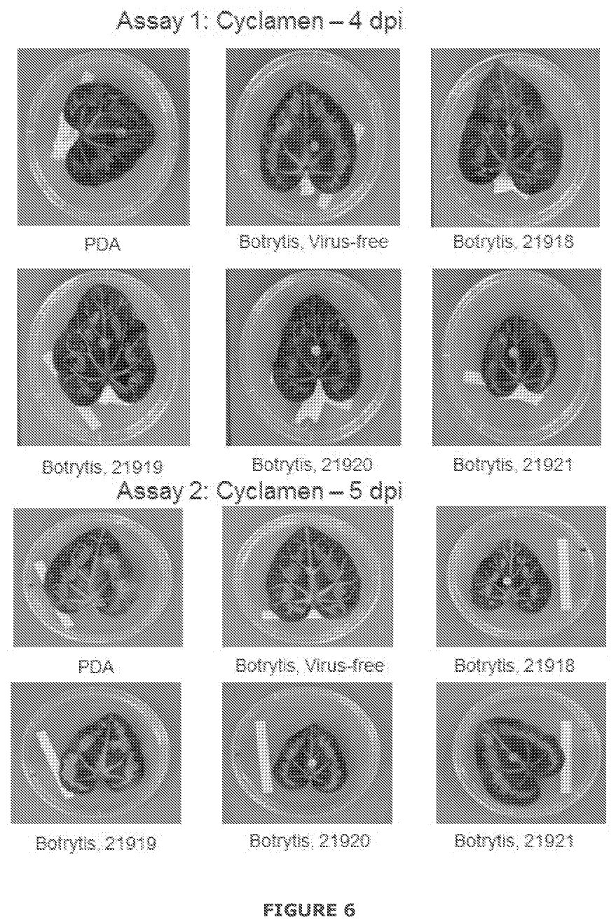

Preliminary hypovirulence biological assays were performed on cyclamen leaves (two replicates of 3 leaves per assay), strawberry leaves (two replicates of 3 leaves per assay), grape berries (two replicates of 3 berries per assay) and kiwifruit leaves (one replicate of 3 leaves; assay 2 only, none used in assay 1). Some level of variation was observed between biological replicants and experiment blocks as shown in FIG. 6 to FIG. 9 using either PDA, PDA grown B. cinerea (virus free) or virus infected (Botrytis isolates 21918, 21919, 21920, and 21921).

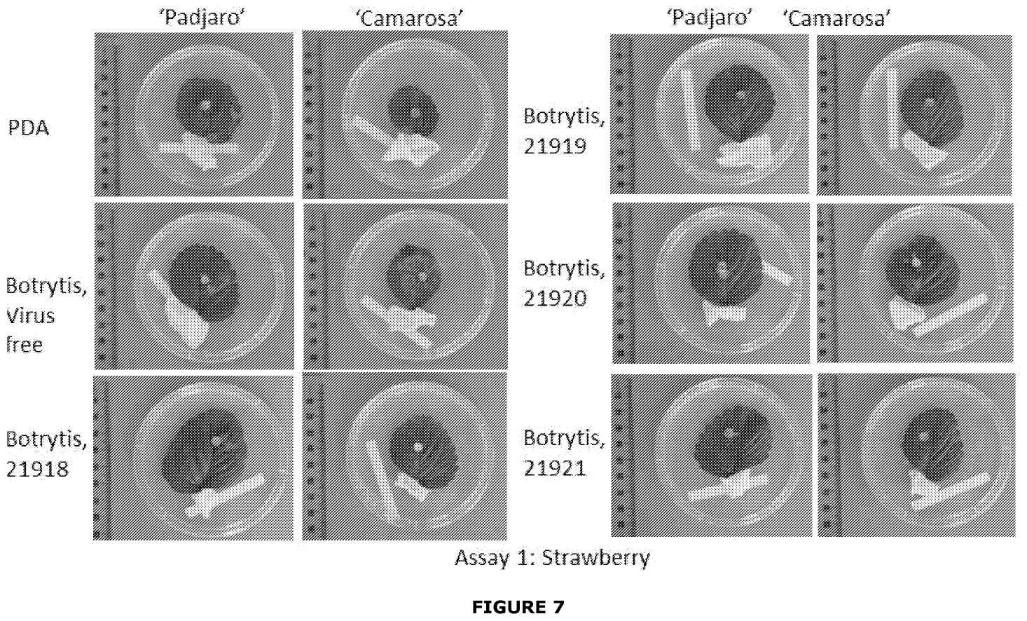

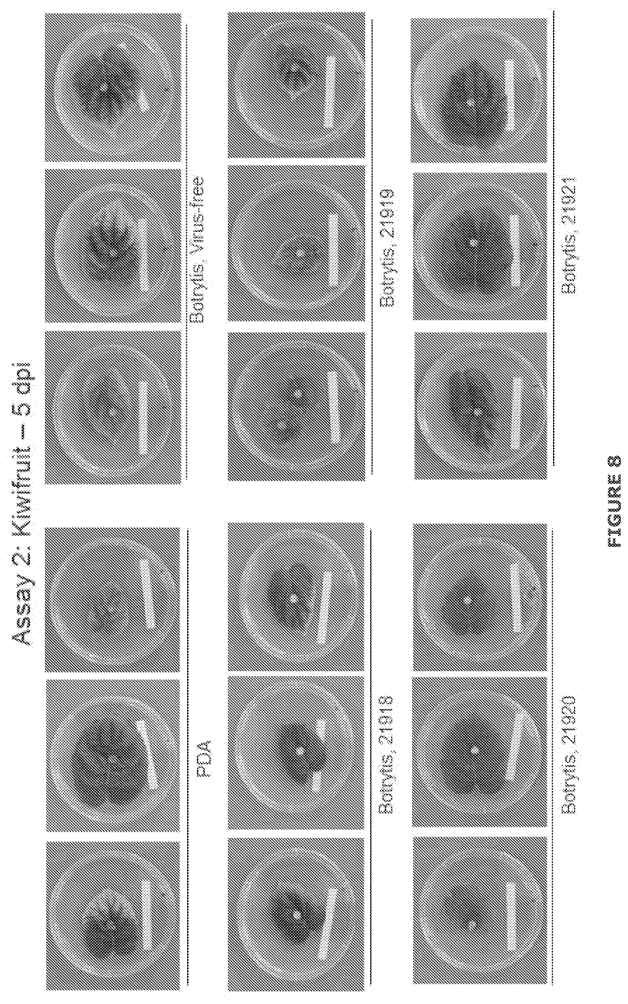

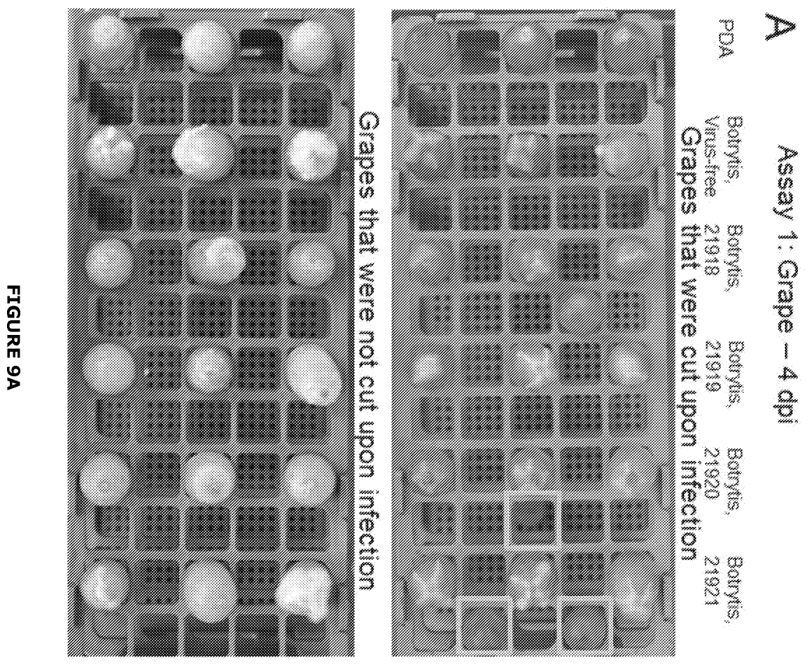

On cyclamen leaves in Assay 1, BGDaV1 infected B. cinerea resulted in slower growth than virus-free B. cinerea (FIG. 6, upper). Similar results occurred for both replicates with strawberry leaves (FIG. 7). However, upon repeat of the experiment (Assay 2) the virus-free B. cinerea did not grow well resulting in poor efficacy on cyclamen leaves (FIG. 6, lower), kiwifruit leaves (FIG. 8) and strawberry leaves (data not shown).

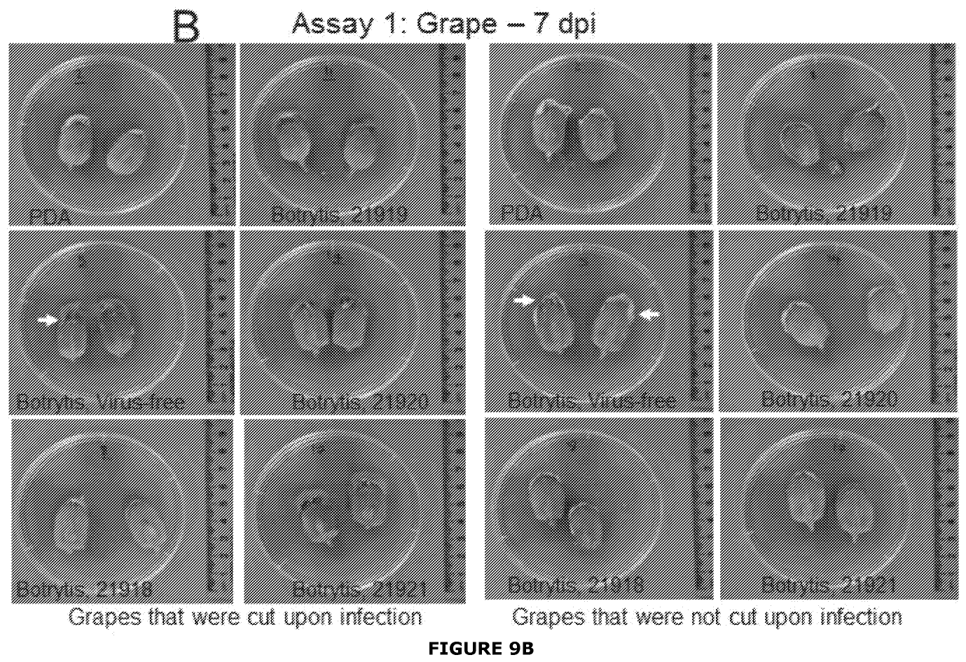

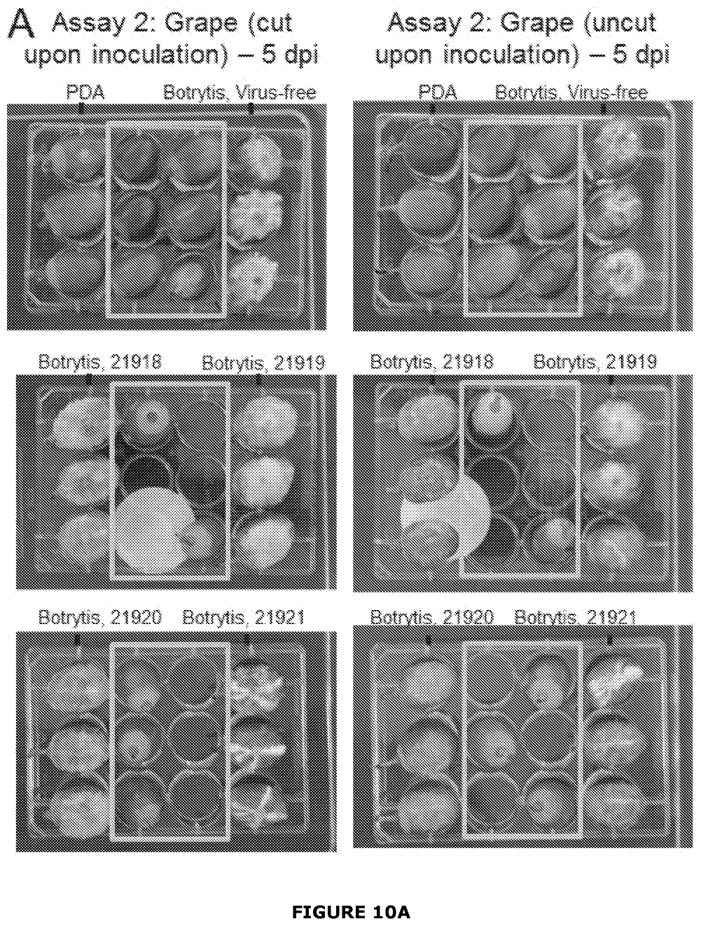

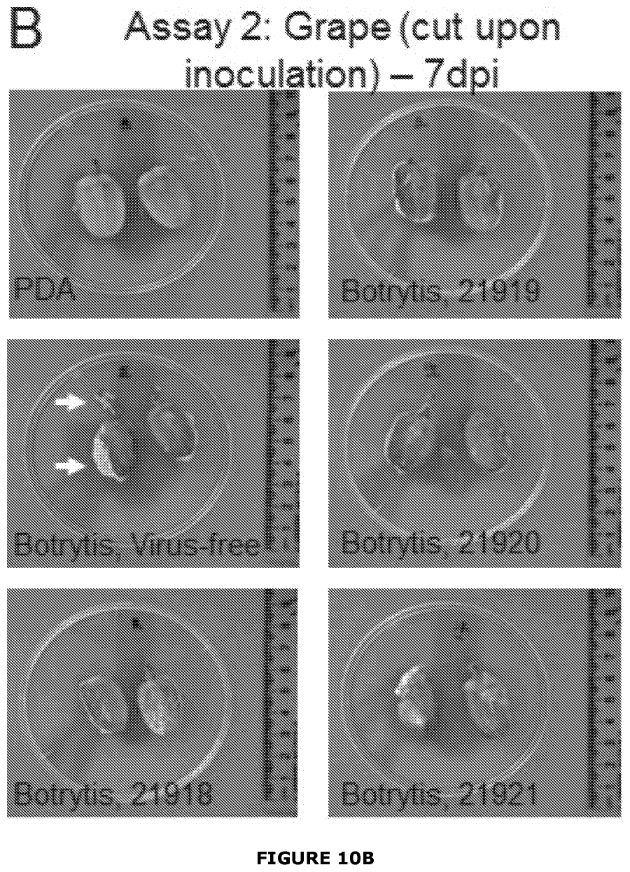

On table grapes in Assay 1, BGDaV1 infected B. cinerea resulted in slower growth than virus-free B. cinerea, particularly when the table grapes were not pre-cut and isolates were infected with BGDaV1 21918 at 4 dpi (FIG. 9). Furthermore, when the grapes were cut in half seven dpi, the grapes inoculated with the virus-free B. cinerea isolate generally had significant loose grape integrity (a classic indicator of Botrytis soft rot), grapes were softer and as shown in FIG. 9B the grapes are considerably misshaped compared with grapes inoculated with BGDaV1-infected B. cinerea that were harder and retained their shape. Upon replication of the experiment (Assay 2) this observed trend was less pronounced (FIG. 10). Growth on the outer surface on the grapes appeared uniform for both table grape assays. Virus-free B. cinerea inoculated grapes still lost shape when grapes were cut in half compared with those inoculated with virus infected B. cinerea (Botrytis isolates 21918, 21919, and 21920). Notably, there was some variability between biological replicates within each assay replication.

### Discussion

In the set of experiments above the inventors demonstrate that under certain conditions, B. cinerea infected with the BGDaV1 virus was efficacious at controlling the Botrytis virulence against winegrapes (berries), and additional fruit or flower crops including kiwifruit, strawberry, and cyclamen. The reasons for the differences observed between replicates, and in particular between individual treatments within Assay 2 are not fully understood. Without wishing to be bound by theory, the inventors believe that the differences may be due to the age of B. cinerea mycelium sampled and/or BGDaV1 distribution within the inoculation source plates. However, irrespective of these differences, the set of reported experiments here demonstrates that BGDaV1 is effective at reducing the virulence of B. cinerea on five important host plants that are typically infected with this fungal pathogen.

Without wishing to be bound by theory the inventors believe that the results of their trials demonstrate that effective biocontrol of phytopathogenic fungi can be achieved using DNA mycoviruses as described herein and that based on the disclosure of the present specification, a skilled worker can readily carry out such biocontrol.

### INDUSTRIAL APPLICATION

The invention has industrial application in being useful for the biocontrol of phytopathogenic fungi, particularly Botrytis spp., particularly B. cinerea.

### REFERENCES

Altschul S F, Gish W, Miller W, Myers E W, Lipman D J 1990. Basic local alignment search tool. Journal of molecular biology, 215(3), 403-410.
Beresford R M 2005. Reducing the impact of fruit spoilage diseases on New Zealand's fruit export industries. New Zealand Plant Protection, 58, 37-41.
Boland G J 1992. Hypovirulence and double-stranded RNA in Sclerotinia sclerotiorum. Canadian Journal of Plant Pathology, 14(1), 10-17. doi:10.1080/07060669209500900
Buck K W 1986. Fungal Virology—An overview. In K. W. Buck (Ed.), Fungal Virology (pp. 1-84). Boca Raton, Fla., USA: CRC Press.
Buck K W 1998. Molecular variability of viruses of fungi. In: Bridge P, Couteaudier Y, Clarkson J eds, Molecular variability of fungal pathogens. Wallingford, UK: CAB International Pp. 53-72.
Bulit J, Dubos B 1988. Botrytis bunch rot and blight. Compendium of grape diseases, 13-15,
Chen B, Chen C-H, Bowman B H, Nuss D L 1996. Phenotypic changes associated with wild-type and mutant hypovirus RNA transfection of plant pathogenic fungi phylogenetically related to Cryphonectria parasitica. Phytopathology, 86(3), 301. doi:10.1094/Phyto-86-301
Chu Y M, Jeon J J, Yea S J, Kim Y H, Yun S H, Lee Y W, Kim K H 2002. Double-stranded RNA mycovirus from Fusarium graminearum. Applied and Environmental Microbiology, 68(5), 2529-2534. doi:10.1128/aem.68.5.2529-2534.2002
Dayaram A, Potter K A, Pailes R, Marinov M, Rosenstein D D, Varsani A 2015. Identification of diverse circular single-stranded DNA viruses in adult dragonflies and damselflies (Insecta: Odonata) of Arizona and Oklahoma, USA. Infection, Genetics and Evolution, 30, 278-287,
Edgar R C 2004. MUSCLE: multiple sequence alignment with high accuracy and high throughput Nucleic Acids Res. 32(5):1792-1797
Elad Y, Malathrakis N E, Dik A J 1996. Biological control of Botrytis-incited diseases and powdery mildews in greenhouse crops, Crop protection, 15(3), 229-240.
Elad Y, Williamson B, Tudzynski P, Delen N. eds. 2004. Botrytis: biology, pathology and control. Dordrecht: Kluwer Academic Publishers.
Elmer P A G, Michailides T J 2007. Epidemiology of Botrytis cinerea in orchard and vine crops. In: Elad Y, Williamson B, Tudzynski P, Delen N eds, Botrytis: Biology, Pathology and Control. Springer, Dordrecht, The Netherlands. Pp. 243-272.
Ghabrial S A 1994. New developments in fungal virology. Advances in Virus Research, 43, 303-388. doi:10.1016/50065-3527(08)60052-4
Ghabrial S A 1998. Origin, adaptation and evolutionary pathways of fungal viruses. Virus Genes, 16(1), 119-131. doi:10.1023/a:1007966229595
Ghabrial S A, Soldevila A I, Havens W M 2002. Molecular genetics of the viruses infecting the plant pathogenic fungus Helminthosporium victoriae. In: Tavantzis S ed. Molecular biology of double-stranded RNA: Concepts and applications in agriculture, forestry and medicine. Boca Raton, Fla., USA: CRC Press. Pp. 213-236.
Goecks 3, Nekrutenko A, Taylor J 2010. Galaxy: a comprehensive approach for supporting accessible, reproducible, and transparent computational research in the life sciences. Genome Biology, 11(8), R86.
Jarvis W R 1977. Botryotinia and Botrytis species: taxonomy, physiology, and pathogenicity (No. 15).
Kanematsu S, Sasaki A, Onoue M, Oikawa Y, Ito T 2010. Extending the fungal host range of a partitivirus and a mycoreovirus from Rosellinia necatrix by inoculation of protoplasts with virus particles. Phytopathology, 100(9), 922-930.
Kearse M, Moir R, Wilson A, Stones-Havas S, Cheung M, Sturrock S. Buxton S, Cooper A, Markowitz S, Duran C, Thierer T, Ashton B, Mentjies P, Drummond A 2012. Geneious Basic: an integrated and extendable desktop software platform for the organization and analysis of sequence data. Bioinformatics, 28(12), 1647-1649,
Khalifa M E, Pearson M N 2013. Molecular characterization of three mitoviruses co-infecting a hypovirulent isolate of Sclerotinia sclerotiorum fungus. Virology, 441(1), 22-30. doi:10.1016/j.viro1.2013.03.002
Khalifa M E, Pearson M N 2014. Characterisation of a novel hypovirus from Scierotinia sclerotiorum potentially representing a new genus within the Hypoviridae. Virology, 464, 441-449.
Khalifa and MacDiarmid, 2017. Mechanical transmitted DNA virus control of Botrytis. CRC2126.
Kraberger S, Arguello-Astorga G R, Greenfield L G, Galilee C, Law D, Martin D P, Varsani A 2015a. Characterisation of a diverse range of circular replication associated protein encoding DNA viruses recovered from a sewage treatment oxidation pond. Infection, Genetics and Evolution, 31, 73-86.
Kraberger S, Farkas K, Bernardo P, Booker C, Arguello-Astorga G R, Mesleard F, Martin D P, Roumagnac P, Varsani A 2015b. Identification of novel Bromus- and Trifolium-associated circular DNA viruses. Archives of Virology, 160 (5), 1303-1311.
Kraberger S, Stainton D, Dayaram A, Zawar-Reza P, Gomez C, Harding J S, Varsani A 2013. Discovery of Sclerotinia sclerotiorum Hypovirulence-Associated Virus-1 in urban river sediments of Heathcote and Styx rivers in Christchurch city, New Zealand. Genome Announcements, 1(4). doi:10.1128/genomeA.00559-13
Kumar S, Stecher G, Tamura K 2016. MEGA7: Molecular Evolutionary Genetics Analysis version 7.0 for bigger datasets. Molecular Biology and Evolution doi: 10.1093/molbev/msw054
Ng T F F, Milner D L, Lim Y W, Schmieder R, Chau B, Nilsson C, Anthony S, Ruan Y, Rohwer F, Breitbart M 2011. Broad surveys of DNA viral diversity obtained through viral metagenomics of mosquitoes, PLoS One, 6(6), e20579.
Nuss D L, Koltin Y 1990. Significance of dsRNA genetic elements in plant pathogenic fungi. Annual Review of Phytopathology, 28, 37-58. doi:10.1146/annurev.py.28.090190.000345
Pearson M N, Beever R E, Boine B, Arthur K 2009. Mycoviruses of filamentous fungi and their relevance to plant pathology. Molecular Plant Pathology, 10(1), 115-128. doi:10.1111/j.1364-3703.2008.00503.x
Pennycook S R 1989. Plant diseases recorded in New Zealand. Volumes 1, 2 and 3. Plant Diseases Division, DSIR.
Rocha J R S, Oliveira N T 1998. Controle biológic de Colletotrichum gloeosporioides, agente da antracnose do maracujazeiro (Passiflora edulis), corn Trichoderma koningii. Summa Phytopathologica, 24, 180-183.
Varga J, Toth B, Vagvolgyi C 2003. Recent advances in mycovirus research. Acta Microbiologica et Immunologica Hungarica, 50(1), 77-94. doi:10.1556/AMicr.50.2003.1.8.
Varsani A, Krupovic M. 2017. Sequence-based taxonomic framework for the classification of uncultured single-stranded DNA viruses of the family Genomoviridae. Virus Evolution 3(1):vew037.
Williamson B, Tudzynski B, Tudzynski P, Van Kan J A 2007. Botrytis cinerea: the cause of grey mould disease. Molecular Plant Pathology 8(5), 561-580. doi: 10.1111/j.1364-3703.2007.00417.

What we claim is:

1. An isolated DNA mycovirus or degenerate strain thereof encoding at least one polypeptide comprising at least 70% sequence identity to a polypeptide selected from the group consisting of SEQ ID NO: 2, SEQ ID NO: 4 and SEQ ID NO: 6.

2. An isolated polypeptide comprising at least 70% sequence identity to an amino acid sequence selected from the group consisting of SEQ ID NO: 2, SEQ ID NO: 4 and SEQ ID NO: 6.

3. An isolated nucleic acid sequence encoding at least one polypeptide of claim 1 or claim 2.

4. An isolated nucleic acid sequence comprising at least 70% sequence identity to SEQ ID NO: 1.

5. An isolated DNA mycovirus comprising SEQ ID NO: 1, or a degenerate strain thereof.

6. A vector comprising an isolated nucleic acid sequence of claim 4.

7. An isolated host cell comprising a nucleic acid sequence of claim 3 or claim 4, an isolated DNA mycovirus of claim 5, or a vector of claim 6.

8. The isolated host cell of claim 7 that is a cell of a hypovirulent fungal strain.

9. A composition comprising an isolated DNA mycovirus of claim 1 or claim 5, an isolated polypeptide of claim 2, an isolated nucleic acid sequence of claim 3 or claim 4, a vector of claim 6 or an isolated host cell of claim 7 or claim 8, or a combination thereof, and a carrier, diluent or excipient.

10. A method of reducing the virulence of at least one phytopathogenic fungus comprising contacting the fungus with an isolated DNA mycovirus of claim 1 or claim 5, a vector of claim 6, an isolated cell of claim 7 or claim 8, a composition of claim 9 or a combination thereof.

11. A method of Botrytis spp. biocontrol comprising contacting at least one Botrytis spp. with an isolated DNA mycovirus, or degenerate strain thereof.

12. A method of treating at least one plant disease caused by a phytopathogenic fungus comprising contacting the plant with an isolated DNA mycovirus of claim 1 or claim 5, a vector of claim 6, an isolated host cell of claim 7 or claim 8, a composition of claim 9, or a combination thereof.

13. A method of controlling at least one phytopathogenic fungus comprising contacting the fungus with an isolated DNA mycovirus of claim 1 or claim 5, a vector of claim 6, an isolated host cell of claim 7 or claim 8, a composition of claim 9, or a combination thereof. The method of claim 10, 12 or 13 wherein the phytopathogenic fungus is a Botrytis spp., preferably Botrytis cinerea.

14. An isolated DNA mycovirus or degenerate strain thereof of claim 1 or claim 5 for use in controlling at least one phytopathogenic fungal strain.

15. An isolated hypovirulent fungal cell or part thereof of claim 8 for use in controlling at least one phytopathogenic fungal strain.

16. The isolated DNA mycovirus or degenerate strain thereof of claim 14 or the isolated hypovirulent fungal cell or part thereof of claim 15, wherein the phytopathogenic fungal strain is Botrytis spp., preferably Botrytis cinerea.

17. An isolated DNA mycovirus, or a degenerate strain thereof, for use in controlling Botrytis spp. fungi.
